(19) United States
(12) Patent Application Publication
(10) Pub. No.: US 2025/0271424 A1
(43) Pub. Date: Aug. 28, 2025

(54) TARGET INTERFERENCE SUPPRESSED ANTI-DRUG ANTIBODY ASSAY

(71) Applicant: Hoffmann-La Roche Inc., Little Falls, NJ (US)

(72) Inventors: Uwe Dahl, Mannheim (DE); Gregor Jordan, Gröbenzell (DE); Roland Staack, Muenchen (DE); Miriam Moheysen-Zadeh, Penzberg (DE)

(21) Appl. No.: 18/946,186

(22) Filed: Nov. 13, 2024

Related U.S. Application Data
(63) Continuation of application No. 16/763,435, filed on May 12, 2020, now Pat. No. 12,174,184.

(30) Foreign Application Priority Data
Nov. 29, 2017 (EP) 17204316.8

(51) Int. Cl. G01N 33/543; G01N 33/53

(52) U.S. Cl. CPC G01N 33/54393; G01N 33/53; G01N 2400/10

(57) ABSTRACT

Herein is reported an immunoassay for quantifying the amount of anti-drug antibody, which anti-drug antibody can specifically bind to a drug antibody, which drug antibody can specifically bind to a therapeutic target, in a scrum or plasma sample comprising the steps of a) incubating the serum or plasma sample at a pH value that is about the pI value of the target, and optionally removing formed precipitate after the incubation, b) incubating the serum or plasma sample obtained in step a) at a pHI value of about 2, and optionally centrifuging the incubated sample to remove formed precipitate, c) adjusting the pH value to about 7.4, adding capture antibody conjugated to a first member of a binding pair and tracer antibody conjugated to a detectable label to the serum or plasma sample obtained in step b) and incubating the mixture to form a capture antibody-anti-drug antibody-tracer antibody-complex, d) quantifying the complex formed in step c) and thereby quantifying the amount of anti-drug antibody in the serum or plasma sample.

### CROSS-REFERENCE TO RELATED APPLICATIONS

This application is a Continuation of U.S. application Ser. No. 16/763,435, filed May 12, 2020, which is a National Stage Application of PCT/EP2018/082664, filed Nov. 27, 2018, which claims priority from European Patent Application No. 17204316.8, filed on Nov. 29, 2017. The priority of said PCT and European Patent Application are claimed. Each of prior mentioned applications is hereby incorporated by reference herein in its entirety.

### REFERENCE TO AN ELECTRONIC SEQUENCE LISTING

The instant application contains a Sequence Listing which has been submitted electronically in XML file format and is hereby incorporated by reference in its entirety. Said XML copy, created on Jan. 27, 2025, is named 107793-103303_P34384-US1_SL.xml and is 156,539 bytes in size.

The current invention is in the field of anti-drug antibody assays. Herein is reported an anti-drug antibody assay with reduced interference from the target of the therapeutic drug.

### BACKGROUND OF THE INVENTION

Moxness, M., et al. (Ann. N. Y. Acad. Sci. USA 1005 (2003) 265-268) reported a radioligand binding assays for total and Ig classes of insulin antibodies (IAB). Test and control sera first were acidified to dissociate bound insulin, and charcoal was added to adsorb the serum insulin. After neutralization, the charcoal with bound insulin was removed from the serum by centrifugation. For each assay, insulin-extracted serum samples were incubated with radiolabeled insulin in the presence and absence of high levels of unlabeled insulin to determine nonspecific binding and total binding, respectively. Thus, Moxness et al. reported a comparison of two ADA assay protocols wherein overnight incubation and acid dissociation were compared.

Patton, A., et al. (J. Immunol. Meth. 304 (2005) 189-195) reported a bridging ELISA that uses a covalently coupled high density antigen surface combined with an acid dissociation step to allow for antibody detection in the presence of antigen in human serum, i.e. without prior removal of excess antigen. Thus, Patton et al. reported an assay protocol in which the excess antigen is not removed prior analysis of the therapeutic antibody. The authors compare the acid pretreated samples with non-pretreated samples, but otherwise identical assay procedure.

Lee, J. W., et al. (AAPS J. 13 (2011) 99-110) report that the predominant driver of bioanalysis in supporting drug development is the intended use of the data. Reliable methodologies for measurements of mAb and its antigen ligand (L) in circulation are crucial for the assessment of exposure-response relationships in support of efficacy and safety evaluations, and dose selection. Ligand-binding assays (LBA) are widely used for the analysis of protein biotherapeutics and antigen ligands (L) to support pharmacokinetics/pharmacodynamics (PK/PD) and safety assessments. For monoclonal antibody drugs (mAb), in particular, which non-covalently bind to L, multiple forms of mAb and L can exist in vivo, including free mAb, free L, and mono- and/or bivalent complexes of mAb and L. Given the complexity of the dynamic binding equilibrium occurring in the body after dosing and multiple sources of perturbation of the equilibrium during bioanalysis, it is clear that ex vivo quantification of the forms of interest (free, bound, or total mAb and L) may differ from the actual ones in vivo. LBA reagents and assay formats can be designed in principle to measure the total or free forms of mAb and L. However, confirmation of the forms being measured under the specified conditions can be technically challenging.

Kelly, M., et al. (AAPS J., 15 (2013) 646-658) report that one area that has been getting increasing attention recently is in the assessment of “free” and “total” analyte and the impact of the assay format on those assessments. The authors provide a critical review of available literature and prospectively explore methods to mitigate the potential impact of anti-drug antibody on PK assay measurement. Furthermore, the methods for increasing drug tolerance in ADA (anti-drug antibody) assays could be re-purposed for assessing or increasing ADA tolerance in PK assays, usually with a preparatory step to break up the immune complex and extract the drug. It must be noted that implementation of such challenging manipulations would not be considered routine for late-stage clinical bioanalysis, but would provide valuable information early on in the investigative stage of method development to pharmacokinetics for their interpretation. Ultimately, any extraction process used to help quantitate drug would likely result in a “total” assessment.

Davis, R. A., et al. (J. Pharm. Biomed. Anal. 48 (2008) 897-901) reported a method for quantifying total (free plus bound) biomarker concentration in the presence of high levels of therapeutic MoAb using a single non-competing MoAb in a capture/acid elution format. This assay has the capability to accurately detect and quantitate circulating ng/ml biomarker levels in the presence of 200 μ/ml or more of therapeutic MoAb.

Salimi-Moosavi, H., et al. (J. Pharm. Biomed. Anal. 51 (2010) 1128-1133) reported alkaline and acid/guanidine treatment approaches to dissociate the protein binding and preferentially denature the ThA. The neutralized antigen proteins can be determined by ELISA. These methods provide reproducible measurements of total antigen protein without ThA interference. Serum samples, standards and QCs containing antigen protein and ThA were treated with alkaline buffer (pH>13) containing casein or acid/guanidine buffer (pH<1). Total antigen proteins for two different ThA systems were successfully measured and interferences were completely eliminated by the treatments. These methods were successfully applied to analysis in pre-clinical serum samples.

Smith, H. W., et al. (Regul. Toxicol. Pharmacol. 49 (2007) 230-237) disclosed the detection of antibodies against therapeutic proteins in the presence of residual therapeutic protein using a solid-phase extraction with acid dissociation (SPEAD) sample treatment prior to ELISA.

An affinity capture elution (ACE) assay for detection of anti-drug antibody to monoclonal antibody therapeutics in the presence of high levels of drug was disclosed by Bourdage, J. S., et al. (J. Immunol. Meth. 327 (2007) 10-17).

Zoghbi, J., et al. (J. Immunol. Meth. 426 (2015) 62-69 disclosed a breakthrough novel method to resolve the drug and target interference problem in immunogenicity assays comprising four components for detection of total ADA (free ADA and drug bound ADA) in the presence of drug in patient samples: (1) use excess drug to saturate free ADA to form drug bound ADA as drug:ADA complexes, (2) precipitate the complex using an agent such as PEG, (3) acid dissociate ADA from drug and immobilize (capture) free ADA (and free drug) under acidic conditions (without neutralization) onto a large capacity surface, and (4) detect free ADA (not the captured drug) using specific anti-human Ig detection reagent.

An affinity capture elution (ACE) assay for detection of anti-drug antibody to monoclonal antibody therapeutics in the presence of high levels of drug was disclosed by Bourdage, J. S., et al. (J. Immunol. Meth. 327 (2007) 10-17).

The current anti-drug antibody (ADA) assay gold standard is the bridging assay with the drug on both sides of a formed complex which is detected. This seems to be the appropriate assay format to detect ADA isotypes and ADA specificity.

Collet-Brose, J., et al., (J. Immunol. Res., Article ID 5069678 (2016)) disclosed the evaluation of multiple immunoassay technology platforms to select the anti-drug antibody assay exhibiting the most appropriate drug and target tolerance. The aim of this study was, at the assay development stage and thus with an appropriate degree of rigor, to select the most appropriate technology platform and sample pretreatment procedure for a clinical ADA assay.

WO 2008/031532 disclosed an antibody binding specifically to Cynomolgus IgG characterized by not binding to Human IgG, and a method for the immunological determination of an immune complex (DA/ADA complex) of a drug antibody (DA) and an antibody against said drug antibody (anti-drug antibody, ADA) in a sample of a monkey species using a double antigen bridging immunoassay. Herein a specific anti-cynomolgus monkey IgG that does not cross bind to human IgG is used, whereby this assay can't be used to analyze human samples. Additionally, the presence of soluble therapeutic target will not result in a false positive signal in the assay as reported in WO 2008/031532.

WO 2015/123315 disclosed assays for detecting the presence or amount of an anti-drug antibody comprising a precipitation step resulting in a precipitation of immune-complexes (drug with ADA) followed by an acidification of the precipitate resulting in a release of ADA from the complexes and an acidic adsorption of the ADA to a surface for a final setup of measurable complexes.

Llinares-Tello, F., et al. (BMJ 73 (2014) THU0166) disclosed the usefulness of the acid dissociation in immunogenicity detection in patients in treatment with anti-TNF drugs in a standard ADA assay.

### SUMMARY OF THE INVENTION

Herein is reported an anti-drug antibody assay wherein the masking of the anti-drug antibody by the (therapeutic) target of the drug is reduced or even eliminated.

Herein is reported an anti-drug antibody assay which is especially useful for samples comprising the drug, its target and anti-drug antibodies, wherein the interference of the (therapeutic) target of the drug is reduced or even eliminated.

The invention is based, at least in part, on the finding that an incubation step performed at the pI of the target or at an acidic pH value prior to the detection of the anti-drug antibody can be used to reduce the interference from the target of the therapeutic antibody (drug) present in a serum or plasma sample in an anti-drug antibody (detection) assay. The assay according to the current invention is especially useful either if the target of the therapeutic drug tends to aggregate and causes thereby non-specific binding, or/and if the target is bivalent/multivalent and thereby normally resulting in a false positive signal in the assay.

The invention is based, at least in part, on the finding that interference of soluble (therapeutic) target of the drug (therapeutic antibody) present in the sample to be analyzed in an immunoassay can be reduced or even eliminated by using two or more acid dissociation steps in the assay procedure.

The invention is based, at least in part, on the finding that the precipitation/aggregation properties of the soluble (therapeutic) target can be used in the presence of ADA and drug to reduce interference of the soluble (therapeutic) target in an immunoassay. The generally used acidification to a low pH values sill result in a high (background) signal resulting in a loss in sensitivity (see e.g. FIG. 18). It has been found that to improve the soluble target deactivation step/reduce target interference it is not necessary to include a precipitation of immune-complexes in the assay procedure. It has been found that an improvement in assay sensitivity/reduction of soluble (therapeutic) target interference in an immunoassay can be achieved by acidification and neutralization without any separation and/or resuspension of the precipitate.

The invention is based, at least in part, on the finding that an acid treatment step can be used to remove, i.e. precipitate, the soluble target (e.g. at/near to its iso-electric point). Using said acid treatment step to remove soluble (therapeutic) target in the sample and to reduce target interference in an immunoassay makes the method according to the current invention generally applicable. Without being bound by this theory it is assumed that the method according to the current invention is more suitable for the analysis of clinical samples with unknown immune response as the soluble (therapeutic) target can be assumed to be present at comparable amounts in different individuals.

The invention is based, at least in part, on the finding that an incubation at the pI of the soluble target is advantageous for improving the performance of an immunoassay, e.g. in reducing target interference or in improving assay sensitivity.

One aspect according to the current invention is an immunoassay for detecting and/or determining and/or quantifying the amount of anti-drug antibody, which anti-drug antibody can specifically bind to a drug antibody, which drug antibody can specifically bind to a therapeutic target, in a serum or plasma sample with reduced target interference comprising the following steps (in the following order):
a) incubating an immobilized capture antibody with a serum or plasma sample comprising drug, target and anti-drug antibody, to form a capture antibody-anti-drug antibody complex,
b) washing the complex formed in step a) with a buffer comprising a sugar and a detergent, which has a pH value of about the pI of the target,
c) incubating for 12 to 24 hours the washed complex of step b) with a tracer antibody conjugated to a (detectable) label to form a capture antibody-anti-drug antibody-tracer antibody complex, (and)
d) detecting and/or determining and/or quantifying the amount of anti-drug antibody by determining the (detectable) label in the complex formed in step c).

In one embodiment the drug is an antibody (drug antibody).

In one embodiment the tracer antibody and the capture antibody is the drug antibody.

In one embodiment the immunoassay comprises a capture antibody, a tracer antibody and a detection antibody, wherein the capture antibody is the drug conjugated to a first member of a binding pair, the tracer antibody is the drug antibody conjugated to a detectable label and the detection antibody, which is further conjugated to an enzyme, is an antibody specifically binding to the detectable label.

In one embodiment the capture antibody and/or the tracer antibody is independently of each other selected from the group consisting of complete/full length drug antibody, F(ab′)2, Fab and scFv of the drug antibody. In one embodiment the capture antibody and the tracer antibody are each a full length drug antibody, or a F(ab′)2 of the drug antibody, or a Fab of the drug antibody.

In one embodiment the sugar is a monosaccharide, a disaccharide or a trisaccharide. In one embodiment the sugar is a disaccharide. In one embodiment the sugar is selected from the group of disaccharides consisting of saccharose, lactose, maltose, iso-maltose, and trehalose. In one preferred embodiment the sugar is saccharose.

In one embodiment the sugar has a concentration of about 6.5 wt-%.

In one embodiment the sugar is saccharose at a concentration of about 6.5 wt-%.

In one embodiment the detergent is a non-ionic detergent. In one embodiment the detergent is selected from the group of detergents consisting of polyalkylene glycol ether (trade name Brij), polyoxyethylene sorbitane monoesters (trade name Tween), octylphenol ethoxylate (trade name Trion or Nonident), octyl-beta-glycoside, n-fatty acid-N-methyl-D-glucamide (trade name MEGA), and N,N′-bis-(3-D-gluconamidopropyl) cholamide (tradename CHAP). In one preferred embodiment the detergent is polyethylene glycol dodecyl ether.

In one embodiment the sugar is saccharose and the detergent is polyethylene glycol dodecyl ether.

In one embodiment the incubation is for 14 to 20 hours. In one embodiment the incubating is for 15 to 17 hours. In one embodiment the incubating is for about 16 hours.

In one embodiment the sugar is saccharose, the detergent is polyethylene glycol dodecyl ether and the incubating is for 15 to 17 hours.

In one embodiment the first member of a binding pair is selected from the group consisting of hapten, antigen and hormone. In one embodiment the binding pair is an antigen/antibody pair or a hapten/anti-hapten antibody pair.

In one embodiment the binding pair is selected from the group consisting of biotin/(strept)avidin, theophylline/anti-theophylline antibody, 5-bromo-desoxy-uridine/anti-5-bromo-deoxy-uridine antibody, digoxigenin/anti-digoxygenin antibody, and helicar/anti-helical antibody. In one embodiment the binding pair is biotin and (strept)avidin.

In one embodiment the drug is an anti-C5 antibody and the target is human C5.

In one embodiment the sugar is saccharose, the detergent is polyethylene glycol dodecyl ether, the drug is an anti-C5 antibody, and the target is human C5.

In one embodiment the sugar is saccharose, the detergent is polyethylene glycol dodecyl ether, the drug is an anti-C5 antibody, the target is human C5 and the buffer has a pH value of about 5.5 or about 5.0.

In one embodiment the sample is a human sample (human serum or plasma sample).

One aspect as reported herein is an immunoassay for detecting and/or determining and/or quantifying the amount of anti-drug antibody, which anti-drug antibody can specifically bind to a drug antibody, which drug antibody can specifically bind to a therapeutic target, in a serum or plasma sample with reduced target interference comprising the following steps:
a) incubating the serum or plasma sample at a pH value that is about the pI value of the target, and optionally removing formed precipitate after the incubation,
b) incubating the serum or plasma sample obtained in step a) at a pH value of about 2, and optionally centrifuging the incubated sample to remove formed precipitate,
c) adjusting the pH value to about 7.4, adding capture antibody conjugated to a first member of a binding pair and tracer antibody conjugated to a detectable label to the serum or plasma sample obtained in step b), and incubating the mixture to form a capture antibody-anti-drug antibody-tracer antibody-complex, (and)
d) measuring and/or determining and/or quantifying the complex formed in step c) and thereby detecting and/or determining and/or quantifying the amount of anti-drug antibody in the serum or plasma sample.

In one embodiment the step of measuring and/or determining and/or quantifying the capture antibody-anti-drug antibody-tracer antibody-complex (step d)) comprises the steps of
d1) incubating the serum or plasma sample obtained in step c) with the second member of the binding pair conjugated to a solid surface to capture the capture antibody-anti-drug antibody-tracer antibody-complex, and optionally washing the surface,
d2) detecting and/or determining and/or quantifying the amount of anti-drug antibody by determining the detectable label in the complex formed in step d1).

In one embodiment the incubation at about the pI value of the target is at a pH value in the range of 0.5 pH units below the pI of the target to 0.5 pH units above the pI value of the target.

In one embodiment the incubating in step a) is with agitation.

In one embodiment the incubating in step a) is for 1.5 to 2.5 hours. In one preferred embodiment the incubating in step a) is for about 2 hours.

In one embodiment the incubating in step b) is for about 5 min.

In one embodiment the incubating in step d) is for about 60 min.

In one embodiment the tracer antibody and the capture antibody is the drug antibody.

In one embodiment the immunoassay comprises a capture antibody, a tracer antibody and a detection antibody, wherein the capture antibody is the drug conjugated to a first member of a binding pair, the tracer antibody is the drug antibody conjugated to a detectable label and the detection antibody, which is conjugated to an enzyme, is an antibody specifically binding to the detectable label.

In one embodiment the capture antibody and/or the tracer antibody is independently of each other selected from the group consisting of complete/full length drug antibody, F(ab′)2, Fab and scFv of the drug antibody. In one embodiment the capture antibody and the tracer antibody are each a full length drug antibody, or a F(ab′)2 of the drug antibody, or a Fab of the drug antibody.

In one embodiment the first member of a binding pair is selected from the group consisting of hapten, antigen and hormone. In one embodiment the binding pair is an antigen/antibody pair or a hapten/anti-hapten antibody pair.

In one embodiment the binding pair is selected from the group consisting of biotin/(strept)avidin, theophylline/anti-theophylline antibody, 5-bromo-desoxy-uridine/anti-5-bromo-deoxy-uridine antibody, digoxigenin/anti-digoxygenin antibody, and helicar/anti-helical antibody. In one embodiment the binding pair is biotin and (strept)avidin.

In one embodiment the drug is an anti-C5 antibody and the target is human C5. In one embodiment the pH value in step a) is in the range of pH 4.7 to pH 5.5. In one preferred embodiment the pH value in step a) is about pH 5.0 or about pH 5.5.

In one embodiment the immunoassay for detecting and/or determining and/or quantifying the amount of anti-drug antibody, which anti-drug antibody can specifically bind to a drug antibody, which drug antibody is an anti-C5 antibody that can specifically bind to human C5, in a serum or plasma sample with reduced target interference comprises the following steps:
a) incubating the serum or plasma sample at a pH value in the range of 4.7 to 5.5 for 1.5 to 2.5 hours, and optionally removing formed precipitate after the incubation,
b) incubating the serum or plasma sample obtained in step a) at a pH value of about 2 for about 5 minutes, and optionally centrifuging the incubated sample to remove formed precipitate,
c) adjusting the pH value to about 7.4, adding capture drug antibody conjugated to biotin and tracer drug antibody conjugated to digoxigenin to the serum or plasma sample obtained in step b) and incubating the mixture to form a capture antibody-anti-drug antibody-tracer antibody-complex,
d) incubating the serum or plasma sample obtained in step c) with (strept)avidin conjugated to a solid surface to capture the capture antibody-anti-drug antibody-tracer antibody-complex, and optionally washing the surface, (and)
e) detecting and/or determining and/or quantifying the amount of anti-drug antibody by determining the digoxigenin in the complex formed in step d) by incubating with an anti-digoxigenin antibody conjugated to horseradish peroxidase and thereafter incubation with HPPA or TMB, and thereby detecting and/or determining and/or quantifying the amount of anti-drug antibody in the serum or plasma sample (correlating the formed complex to the amount of the ADA in the sample).

In one embodiment of all aspects the sample is from an animal. In one embodiment the animal is selected from a human being and an experimental animal. In one embodiment the sample is from an animal to which the drug had been administered prior to obtaining the sample. In one embodiment the sample is from a patient in need of a treatment with the drug to which the drug had been administered prior to obtaining the sample. In no case is the sample re-applied to a living being after the method as reported herein had been performed therewith.

In one embodiment of all aspects the sample is a human sample (human serum or plasma sample).

In one embodiment of all aspects the complexes are non-covalent complexes.

Generally, an immunoassay comprises the following steps:
a) immobilizing the capture antibody on a solid surface, and optionally washing the surface after the immobilization step to remove unbound and non-specifically bound capture antibody,
b) incubating the immobilized capture antibody of step a) with a serum or plasma containing sample, which optionally has been diluted to have a concentration of the anti-drug antibody within the detection range of the immunoassay, to form a capture antibody-anti-drug antibody-complex, and optionally washing the surface after the incubation step to remove unbound and non-specifically bound sample,
c) incubating the capture antibody-anti-drug antibody-complex of step b) with a labelled tracer antibody to form a capture antibody-anti-drug antibody-tracer antibody complex, and optionally washing the surface after the incubation step to remove unbound and non-specifically bound tracer antibody,
d) incubating the capture antibody-anti-drug antibody-tracer antibody complex of step c) with an antibody specifically binding to the label of the tracer antibody conjugated to an enzyme to form a capture antibody-anti-drug antibody-tracer antibody-detection antibody complex, and optionally washing the surface after the incubation step to remove unbound and non-specifically bound detection antibody,
e) incubating the capture antibody-anti-drug antibody-tracer antibody-detection antibody complex of step d) with a colorless substrate of the enzyme that upon action of the enzyme on the substrate is converted to a colored reaction product and determining the optical density after a predefined period of time, (and)
f) correlating the optical density determined in step e) with a calibration curve and thereby determining the amount of anti-drug antibody in the sample.

### DETAILED DESCRIPTION OF THE INVENTION

For the analysis of therapeutic antibodies (drug or short D) as well as the respective antibodies against the therapeutic antibody (anti-drug antibody or short ADA) in samples of in vitro or in vivo origin a respective assay is necessary.

The ADA binds to its antigen (in vitro and in vivo), i.e. the therapeutic antibody/drug, and an equilibrium between free ADA and free drug, respectively, as well as mono- and di-complexed drug (assuming a bivalent monospecific drug) is formed. This equilibrium is dynamic, i.e. the change of the concentration of one component taking part in this equilibrium also changes the concentrations of all other components taking part in this equilibrium.

While the fraction of free, i.e. not bound, ADA correlates to the availability of drug for binding and binding capacity of ADA to its antigen, i.e. drug, in vivo, the determination of total ADA can be used to characterize the interaction between ADA and drug.

For full pharmacokinetic evaluation of a drug, e.g., the knowledge of ADA concentration, either free, i.e. drug-binding competent or in complex with the drug, in the systemic circulation is important. Free ADA can be evaluated as potential biomarker.

An assay for determining ADA in a sample can be interfered if the antigen of the drug is present in the sample. For pharmacokinetic evaluation the ADA fraction, which can bind or is bound to the drug, is important.

The terms “therapeutic antibody” and “drug” are used interchangeably herein. These terms are used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

In certain embodiments, the drug is a monospecific antibody. In one embodiment the drug is a monospecific, bivalent antibody. In one preferred embodiment the drug is a monoclonal, monospecific, bivalent antibody.

In certain embodiments, the drug is a multispecific antibody, e.g. a bispecific antibody. Multispecific antibodies are monoclonal antibodies that have binding specificities for at least two different antigens. In certain embodiments, one of the binding specificities is for a first antigen and the other is for a different second antigen. In certain embodiments, bispecific antibodies may bind to two different epitopes of the same antigen. Bispecific antibodies can be prepared as full length antibodies or antibody fragments. In one embodiment the antibody is a bispecific antibody, which specifically binds to a first and a second antigen. In one embodiment the bispecific antibody has i) a first binding specificity that specifically binds to a first antigen or a first epitope on an antigen, and ii) a second binding specificity that specifically binds to a second antigen or a second epitope on the (same) antigen. In one embodiment the second epitope on the same antigen is a non-overlapping epitope. In one embodiment the antibody is a bispecific, bivalent antibody. In one preferred embodiment the antibody is a monoclonal, bispecific, bivalent antibody.

Multispecific antibodies are described in WO 2009/080251, WO 2009/080252, WO 2009/080253, WO 2009/080254, WO 2010/112193, WO 2010/115589, WO 2010/136172, WO 2010/145792, or WO 2010/145793.

The terms “anti-C5 antibody” and “an antibody that (specifically) binds to C5” refer to an antibody that is capable of binding C5 with sufficient affinity such that the antibody is useful as a diagnostic and/or therapeutic agent in targeting C5. In one embodiment, the extent of binding of an anti-C5 antibody to an unrelated, non-C5 protein is less than about 10% of the binding of the antibody to C5. In certain embodiments, an anti-C5 antibody binds to an epitope of C5 that is conserved among C5 from different species. In one preferred embodiment C5 is human C5.

The term “C5”, as used herein, encompasses any native C5 from any vertebrate source, including mammals such as primates (e.g., humans and monkeys) and rodents (e.g., mice and rats). Unless otherwise indicated, the term “C5” refers to a human C5 protein having the amino acid sequence shown in SEQ ID NO: 30 and containing the beta chain sequence shown in SEQ ID NO: 31. The term encompasses “full-length”, unprocessed C5 as well as any form of C5 that results from processing in the cell. The term also encompasses naturally occurring variants of C5, e.g., splice variants or allelic variants. The amino acid sequence of an exemplary human C5 is shown in SEQ ID NO: 30 (“wild-type” or “wt” C5). The amino acid sequence of an exemplary beta chain of human C5 is shown in SEQ ID NO: 31. The amino acid sequences of exemplary MG1, MG2 and MG1-MG2 domains of the beta chain of human C5 are shown in SEQ ID NO: 32, 33, and 34, respectively. The amino acid sequences of exemplary cynomolgus monkey and murine C5 are shown in SEQ ID NO: 35 and 96, respectively. Amino acid residues 1-19 of SEQ ID NOs: 30, 31, 34, 35, and 96 correspond to a signal sequence that is removed during processing in the cell and is thus missing from the corresponding exemplary amino acid sequence. The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The principles of different immunoassays are described, for example, by Hage, D. S. (Anal. Chem. 71 (1999) 294R-304R). Lu, B., et al. (Analyst 121 (1996) 29R-32R) report the orientated immobilization of antibodies for the use in immunoassays. Avidin-biotin-mediated immunoassays are reported, for example, by Wilchek, M., and Bayer, E. A., in Methods Enzymol. 184 (1990) 467-469.

Monoclonal antibodies and their constant domains contain a number of reactive amino acid side chains for conjugating to a member of a binding pair, such as a polypeptide/protein, a polymer (e.g. PEG, cellulose or polystyrol), or an enzyme. Chemical reactive groups of amino acids are, for example, amino groups (lysins, alpha-amino groups), thiol groups (cystins, cysteines, and methionins), carboxylic acid groups (aspartic acids, glutamic acids), and sugar-alcoholic groups. Such methods are e.g. described by Aslam M., and Dent, A., in “Bioconjugation”, MacMillan Ref. Ltd. 1999, pages 50-100.

One of the most common reactive groups of antibodies is the aliphatic 8-amine of the amino acid lysine. In general, nearly all antibodies contain abundant lysine. Lysine amines are reasonably good nucleophiles above pH 8.0 (pKa=9.18) and therefore react easily and cleanly with a variety of reagents to form stable bonds. Amine-reactive reagents react primarily with lysins and the α-amino groups of proteins. Reactive esters, particularly N-hydroxy-succinimide (NHS) esters, are among the most commonly employed reagents for modification of amine groups. The optimum pH for reaction in an aqueous environment is pH 8.0 to 9.0. Isothiocyanates are amine-modification reagents and form thiourea bonds with proteins. They react with protein amines in aqueous solution (optimally at pH 9.0 to 9.5). Aldehydes react under mild aqueous conditions with aliphatic and aromatic amines, hydrazines, and hydrazides to form an imine intermediate (Schiff's base). A Schiff's base can be selectively reduced with mild or strong reducing agents (such as sodium borohydride or sodium cyanoborohydride) to derive a stable alkyl amine bond. Other reagents that have been used to modify amines are acid anhydrides. For example, diethylenetriaminepentaacetic anhydride (DTPA) is a bifunctional chelating agent that contains two amine-reactive anhydride groups. It can react with N-terminal and 8-amine groups of amino acids to form amide linkages. The anhydride rings open to create multivalent, metal-chelating arms able to bind tightly to metals in a coordination complex.

Another common reactive group in antibodies is the thiol residue from the sulfur-containing amino acid cystine and its reduction product cysteine (or half cystine). Cysteine contains a free thiol group, which is more nucleophilic than amines and is generally the most reactive functional group in a protein. Thiols are generally reactive at neutral pH, and therefore can be coupled to other molecules selectively in the presence of amines. Since free sulfhydryl groups are relatively reactive, proteins with these groups often exist with them in their oxidized form as disulfide groups or disulfide bonds. In such proteins, reduction of the disulfide bonds with a reagent such as dithiothreitol (DTT) is required to generate the reactive free thiol. Thiol-reactive reagents are those that will couple to thiol groups on polypeptides, forming thioether-coupled products. These reagents react rapidly at slight acidic to neutral pH and therefore can be reacted selectively in the presence of amine groups. The literature reports the use of several thiolating crosslinking reagents such as Traut's reagent (2-iminothiolane), succinimidyl (acetylthio) acetate (SATA), and sulfosuccinimidyl 6-[3-(2-pyridyldithio) propionamido] hexanoate (Sulfo-LC-SPDP) to provide efficient ways of introducing multiple sulfhydryl groups via reactive amino groups. Haloacetyl derivatives, e.g. iodoacetamides, form thioether bonds and are also reagents for thiol modification. Further useful reagents are maleimides. The reaction of maleimides with thiol-reactive reagents is essentially the same as with iodoacetamides. Maleimides react rapidly at slight acidic to neutral pH.

Another common reactive group in antibodies are carboxylic acids. Antibodies contain carboxylic acid groups at the C-terminal position and within the side chains of aspartic acid and glutamic acid. The relatively low reactivity of carboxylic acids in water usually makes it difficult to use these groups to selectively modify polypeptides and antibodies. When this is done, the carboxylic acid group is usually converted to a reactive ester by the use of a water-soluble carbodiimide and reacted with a nucleophilic reagent such as an amine, hydrazide, or hydrazine. The amine-containing reagent should be weakly basic in order to react selectively with the activated carboxylic acid in the presence of the more highly basic s-amines of lysine to form a stable amide bond. Protein crosslinking can occur when the pH is raised above 8.0.

Sodium periodate can be used to oxidize the alcohol part of a sugar within a carbohydrate moiety attached to an antibody to an aldehyde. Each aldehyde group can be reacted with an amine, hydrazide, or hydrazine as described for carboxylic acids. Since the carbohydrate moiety is predominantly found on the crystallizable fragment region (Fc-region) of an antibody, conjugation can be achieved through site-directed modification of the carbohydrate away from the antigen-binding site. A Schiff's base intermediate is formed, which can be reduced to an alkyl amine through the reduction of the intermediate with sodium cyanoborohydride (mild and selective) or sodium borohydride (strong) water-soluble reducing agents.

The conjugation of a tracer and/or capture and/or detection antibody to its conjugation partner can be performed by different methods, such as chemical binding, or binding via a binding pair. The term “conjugation partner” as used herein denotes e.g. solid supports, polypeptides, detectable labels, members of specific binding pairs. In one embodiment the conjugation of the capture and/or tracer and/or detection antibody to its conjugation partner is performed by chemically binding via N-terminal and/or ε-amino groups (lysine), ε-amino groups of different lysines, carboxy-, sulfhydryl-, hydroxyl-, and/or phenolic functional groups of the amino acid backbone of the antibody, and/or sugar alcohol groups of the carbohydrate structure of the antibody. In one embodiment the capture antibody is conjugated to its conjugation partner via a binding pair. In one preferred embodiment the capture antibody is conjugated to biotin and immobilization to a solid support is performed via solid support immobilized avidin or streptavidin. In one embodiment the capture antibody is conjugated to its conjugation partner via a binding pair. In one preferred embodiment the tracer antibody is conjugated to digoxigenin by a covalent bond as detectable label.

The term “sample” includes, but is not limited to, any quantity of a substance from a living thing or formerly living thing. Such living things include, but are not limited to, humans, mice, monkeys, rats, rabbits, and other animals. In one embedment the sample is obtained from a monkey, especially a cynomolgus monkey, or a rabbit, or a mouse, or rat, or a human. In one preferred embodiment the sample is a human sample. Such substances include, but are not limited to, in one embodiment whole blood, plasma or serum from an individual, which are the most widely used sources of sample in clinical routine.

The term “solid phase” denotes a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. A solid phase component is distinguished from inert solid surfaces in that a “solid phase” contains at least one moiety on its surface, which is intended to interact with a substance in a sample. A solid phase may be a stationary component, such as a tube, strip, cuvette or microtiter plate, or may be non-stationary components, such as beads and microparticles. A variety of microparticles that allow either non-covalent or covalent attachment of proteins and other substances may be used. Such particles include polymer particles such as polystyrene and poly (methylmethacrylate); gold particles such as gold nanoparticles and gold colloids; and ceramic particles such as silica, glass, and metal oxide particles. See for example Martin, C. R., et al., Analytical Chemistry-News & Features, 70 (1998) 322A-327A, or Butler, J. E., Methods 22 (2000) 4-23.

Chromogens (fluorescent or luminescent groups and dyes), enzymes, NMR-active groups or metal particles, haptens, e.g. digoxigenin, are examples of “detectable labels”. The detectable label can also be a photoactivatable crosslinking group, e.g. an azido or an azirine group. Metal chelates which can be detected by electrochemiluminescense are also preferred signal-emitting groups, with particular preference being given to ruthenium chelates, e.g. a ruthenium (bispyridyl)₃²⁺ chelate. Suitable ruthenium labeling groups are described, for example, in EP 0 580 979, WO 90/05301, WO 90/11511, and WO 92/14138. For direct detection the labeling group can be selected from any known detectable marker groups, such as dyes, luminescent labeling groups such as chemiluminescent groups, e.g. acridinium esters or dioxetanes, or fluorescent dyes, e.g. fluorescein, coumarin, rhodamine, oxazine, resorufin, cyanine and derivatives thereof. Other examples of labeling groups are luminescent metal complexes, such as ruthenium or europium complexes, enzymes, e.g. as used for ELISA or for CEDIA (Cloned Enzyme Donor Immunoassay, e.g. EP-A-0 061 888), and radioisotopes.

Indirect detection systems comprise, for example, that the detection reagent, e.g., the detection antibody is labeled with a first partner of a binding pair. Examples of suitable binding pairs are antigen/antibody, biotin or biotin analogues such as aminobiotin, iminobiotin or desthiobiotin/avidin or Streptavidin, sugar/lectin, nucleic acid or nucleic acid analogue/complementary nucleic acid, and receptor/ligand, e.g., steroid hormone receptor/steroid hormone. In one preferred embodiment the first binding pair members comprise hapten, antigen and hormone. In one preferred embodiment the hapten is selected from the group consisting of digoxin, digoxygenin and biotin and analogues thereof. The second partner of such binding pair, e.g. an antibody, Streptavidin, etc., usually is labeled to allow for direct detection, e.g., by the labels as mentioned above.

The term “immunoassay” denotes any technique that utilizes specifically binding molecules, such as antibodies, to capture and/or detect a specific target for qualitative or quantitative analysis. In general, an immunoassay is characterized by the following steps: 1) immobilization or capture of the analyte and 2) detection and measuring the analyte. The analyte can be captured, i.e. bound, on any solid surface, such as e.g. a membrane, plastic plate, or some other solid surface.

Immunoassays can be performed generally in three different formats. One is with direct detection, one with indirect detection, or by a sandwich assay. The direct detection immunoassay uses a detection (or tracer) antibody that can be measured directly. An enzyme or other molecule allows for the generation of a signal that will produce a color, fluorescence, or luminescence that allow for the signal to be visualized or measured (radioisotopes can also be used, although it is not commonly used today). In an indirect assay a primary antibody that binds to the analyte is used to provide a defined target for a secondary antibody (tracer antibody) that specifically binds to the target provided by the primary antibody (referred to as detector or tracer antibody). The secondary antibody generates the measurable signal. The sandwich assay makes use of two antibodies, a capture and a trace (detector) antibody. The capture antibody is used to bind (immobilize) analyte from solution or bind to it in solution. This allows the analyte to be specifically removed from the sample. The tracer (detector) antibody is used in a second step to generate a signal (either directly or indirectly as described above). The sandwich format requires two antibodies each with a distinct epitope on the target molecule. In addition, they must not interfere with one another as both antibodies must be bound to the target at the same time.

### Embodiments of the Method According to the Invention

Drug interference in an ADA assay is a generally known phenomenon, but not the interference of the target of the drug in an ADA assay.

Generally, after acid dissociation a neutralization step follows the acid or base dissociation to allow binding partners to bridge anew causing the interference factor if still in solution to re-bind as well, maintaining the issue.

Many approaches have been used to mitigate this problem such as acid or base dissociation, competitive inhibition of interference using specific antibodies, removal of the interference factors, solid phase extraction with acid dissociation (SPEAD), affinity capture elution (ACE) and many others. The use of acid dissociation in a bridging assay has shown some improvement in drug tolerance for the detection of ADA (see e.g. Moxness, M., et al., Clin. Chem. 51 (10), 1983; Patton, A., et al., J. Immunol. Methods 304 (2005) 189).

PEG precipitation of the target molecule or immune complex is size (or molecular weight, MW) based and PEG concentration dependent. The higher the PEG concentrations, the lower MW targets it will precipitate. To reduce the precipitation of non-specific serum proteins such as albumin and immunoglobulin, a low concentration of PEG is used to precipitate large MW drug:ADA immune complexes. Using the principle of precipitation, coupled with acid dissociation and capturing on high capacity surface under acidic conditions (preventing the binding partners from re-binding), allows specific detection of ADA or drug or drug target using specific detection reagents.

Acid dissociation is used commonly to disrupt drug-ADA-complexes and, thus, to release ADA from said immune-complexes. The released (free) ADA can form complexes with the detection antibody in a subsequent step. The acid dissociation step can shorten the overall assay time compared to a classical ADA assay (without acid dissociation step). Generally, the focus is on comparable sensitivity.

In general, a standard ADA assay bears the disadvantage of a long incubation time for forming the new equilibrium between the ADA and the reagents in the presence of residual drug. If a short incubation time is applied only a low drug tolerance can be obtained (=low sensitivity with residual drug).

The formation of the measurable complexes takes time and is depending on the association rate constant of the ADA to the reagents. For the standard ADA assay the dissociation rate constant of the immune-complexes is the time limiting step. For this reason, longer incubation times (commonly overnight) are applied to the incubation of the sample and reagents.

Thus, acid dissociation is predominantly a complex dissociation procedure.

One aspect according to the current invention is an immunoassay for detecting and/or determining and/or quantifying the amount of anti-drug antibody, which anti-drug antibody can specifically bind to a drug antibody, which drug antibody can specifically bind to a therapeutic target, in a serum or plasma sample comprising the following steps:
a) incubating an immobilized capture antibody with a serum or plasma sample comprising drug, target and anti-drug antibody, to form a capture antibody-anti-drug antibody complex,
b) washing the complex formed in step a) with a buffer comprising a sugar and a detergent, which has a pH value of about the pI of the target,
c) incubating for 12 to 24 hours the washed complex of step b) with a labelled tracer antibody to form a capture antibody-anti-drug antibody-tracer antibody complex, (and)
d) detecting and/or determining and/or quantifying the amount of anti-drug antibody by determining the detectable label in the complex formed in step c).

In one embodiment the sugar is saccharose, the detergent is polyethylene glycol dodecyl ether, the drug is an anti-C5 antibody, the target is human C5 and the buffer has a pH value of about 5.5.

One aspect according to the current invention is an immunoassay for detecting and/or determining and/or quantifying the amount of anti-drug antibody, which anti-drug antibody can specifically bind to a drug antibody, which drug antibody can specifically bind to a therapeutic target, in a serum or plasma sample comprising the following steps:
a) incubating the serum or plasma sample at a pH value that is about the pI value of the target, and optionally removing formed precipitate after the incubation,
b) incubating the serum or plasma sample obtained in step a) at a pH value of about 2, and optionally centrifuging the incubated sample to remove formed precipitate,
c) adjusting the pH value to about 7.4, adding capture antibody conjugated to a first member of a binding pair and tracer antibody conjugated to a detectable label to the serum or plasma sample obtained in step b) and incubating the mixture to form a capture antibody-anti-drug antibody-tracer antibody-complex, (and)
d) measuring and/or determining and/or quantifying the complex formed in step c) and thereby detecting and/or determining and/or quantifying the amount of anti-drug antibody in the serum or plasma sample.

In one embodiment the step of measuring and/or determining and/or quantifying the capture antibody-anti-drug antibody-tracer antibody-complex (step d)) comprises the steps of
d1) incubating the serum or plasma sample obtained in step c) with the second member of the binding pair conjugated to a solid surface to capture the capture antibody-anti-drug antibody-tracer antibody-complex, and optionally washing the surface, (and)
d2) detecting and/or determining and/or quantifying the amount of anti-drug antibody by determining the detectable label in the complex formed in step d1).

In one embodiment the immunoassay for detecting and/or determining and/or quantifying the amount of anti-drug antibody, which anti-drug antibody can specifically bind to a drug antibody, which drug antibody is an anti-C5 antibody that can specifically bind to human C5, in a serum or plasma sample comprises the following steps:
a) incubating the serum or plasma sample at a pH value in the range of 4.7 to 5.5 for 1.5 to 2.5 hours, and optionally removing formed precipitate after the incubation,
b) incubating the serum or plasma sample obtained in step a) at a pH value of about 2 for about 5 minutes, and optionally centrifuging the incubated sample to remove formed precipitate,
c) adjusting the pH value to about 7.4, adding capture drug antibody conjugated to biotin and tracer drug antibody conjugated to digoxigenin to the serum or plasma sample obtained in step b) and incubating the mixture to form a capture antibody-anti-drug antibody-tracer antibody-complex,
d) incubating the serum or plasma sample obtained in step c) with (strept)avidin conjugated to a solid surface to capture the capture antibody-anti-drug antibody-tracer antibody-complex, and optionally washing the surface, (and)
e) detecting and/or determining and/or quantifying the amount of anti-drug antibody by determining the digoxigenin in the complex formed in step d) by incubating with an anti-digoxigenin antibody conjugated to horseradish peroxidase and thereafter incubation with HPPA or TMB, and thereby detecting and/or determining and/or quantifying the amount of anti-drug antibody in the serum or plasma sample.

The assay as reported herein addresses the interference of the target of a therapeutic drug in the measurement and/or determination and/or quantification of anti-drug antibodies in a serum or plasma sample.

Normally the interference from drug in the determination and/or measurement and/or quantification of anti-drug antibodies (ADA) in a serum or plasma sample has to be addressed. Measures therefore can be high specific sensitivity for the anti-drug antibody, influencing the equilibrium in the sample towards the free anti-drug antibody, dissociating ADA-drug complexes by sample pre-treatment, detecting ADA-drug complexes, or enriching ADA.

But the interference of the target of the drug in the sample is not addressed thereby.

The immunoassay as reported herein is exemplified in the following with a therapeutic anti-C5 antibody. This is presented merely as an exemplification of the currently reported immunoassay and shall not be construed as a limitation of the current invention.

Human C5 has a serum concentration of approximately 70 μg/ml (approximately 368 nM).

Different sample dilutions (1:100, 1:1000), different capture antibody as well as tracer antibody concentration (500 ng/ml each, 1000 ng/ml each, 1500 ng/ml each, 2000 ng/ml each), different peroxidase concentrations (5 mU, 10 mU, 25 mU, 50 mU at 1000 ng/ml capture and tracer antibody concentration) were evaluated (see FIGS. 1 and 2). For all experiments an overnight sample incubation with reagents was performed.

The invention is based, at least in part, on the finding that the capture antibody and tracer antibody concentration should be at least 500 ng/ml whereby at 1500 ng/ml or more no further signal gain could be achieved.

But a rather high background is present.

In the presence of 1% (v/v) human serum non-specific binding could be observed. The blocking of the plate with BSA did not solve problem of non-specific binding.
without BSA:

| | Bi/Dig | Bi/Dig | —/— | | Bi/— | | —/Dig | | Bi/Dig | |
|---|---|---|---|---|---|---|---|---|---|---|
| | cut-off | individual | neg./pos. relative to buffer cut-off | | | | | | HPS cut-off | |
| HPS | 1631 | 2199 | 66 | 58 | 78 | 78 | 417 | 757 | 4061 | 4070 |
| | | 2198 | 83 | 83 | 68 | 72 | 108 | 126 | 1286 | 1302 |
| | | 2201 | 54 | 60 | 58 | 53 | 74 | 63 | 644 | 669 |
| | | 2195 | 59 | 58 | 53 | 51 | 98 | 88 | 3612 | 3763 |
| buffer | 84 | 2205 | 55 | 75 | 62 | 56 | 89 | 90 | 3591 | 3650 |
| | | 2202 | 53 | 54 | 72 | 51 | 283 | 289 | 5054 | 5200 |
| | | 2194 | 575 | 568 | 302 | 281 | 966 | 1053 | 7336 | 7444 |
| | | 2196 | 129 | 178 | 97 | 119 | 144 | 159 | 1336 | 1379 |

with BSA:

| | Bi/Dig | Bi/Dig | —/— | | Bi/— | | —/Dig | | Bi/Dig | |
|---|---|---|---|---|---|---|---|---|---|---|
| | cut-off | individual | neg./pos. relative to buffer cut-off | | | | | | HPS cut-off | |
| HPS | 1690 | 2199 | 63 | 64 | 75 | 59 | 542 | 709 | 4024 | 3997 |
| | | 2198 | 89 | 96 | 73 | 85 | 112 | 129 | 1294 | 1286 |
| | | 2201 | 55 | 51 | 51 | 51 | 61 | 66 | 653 | 663 |
| | | 2195 | 60 | 54 | 47 | 56 | 79 | 91 | 3589 | 3660 |
| buffer | 71 | 2205 | 51 | 52 | 52 | 52 | 77 | 83 | 3621 | 3621 |
| | | 2202 | 64 | 54 | 59 | 60 | 271 | 265 | 5175 | 5192 |
| | | 2194 | 640 | 620 | 318 | 317 | 1060 | 1060 | 7549 | 7522 |
| | | 2196 | 152 | 157 | 100 | 110 | 152 | 178 | 1370 | 1380 |

It can be seen that even in the absence of the Bi/Dig reagents a high signal is obtained (column “−/−”). Beside others still non-specific binding of detection reagent can be seen (column “−/Dig”).

By the addition of a detergent (Brij 35) a reduction of non-specific binding could be seen (see “−/−”, “Bi/−” and “−/Dig” columns). In the absence of the detergent the cut-point (CP) was 10240 (approx. 2562 ng/ml) (see “Bi/Dig” column), whereas in the presence of the detergent the CP was 4708 (approx. 817 ng/ml) (see “Bi/Dig” column). But the variation coefficient (CV) of the individuals is high.
without detergent:

| | Bi/Dig | Bi/Dig | —/— | | Bi/— | | —/Dig | | Bi/Dig | |
|---|---|---|---|---|---|---|---|---|---|---|
| | cut-off | individual | neg./pos. relative to buffer cut-off | | | | | | HPS cut-off | |
| HPS | 1471 | 2199 | 195 | 198 | 98 | 86 | 992 | 1113 | 7045 | 6993 |
| | | 2198 | 167 | 179 | 66 | 62 | 185 | 209 | 1359 | 1388 |
| | | 2201 | 192 | 157 | 83 | 86 | 189 | 181 | 714 | 749 |
| | | 2195 | 256 | 184 | 63 | 63 | 355 | 393 | 4841 | 4805 |
| buffer | 216 | 2205 | 201 | 192 | 66 | 65 | 370 | 317 | 4431 | 4489 |
| | | 2202 | 153 | 172 | 78 | 86 | 620 | 584 | 6959 | 7110 |
| | | 2194 | 152 | 180 | 78 | 82 | 687 | 807 | 10557 | 10249 |
| | | 2196 | 230 | 243 | 81 | 89 | 241 | 197 | 1410 | 1219 |

with detergent:

| | Bi/Dig | Bi/Dig | —/— | | Bi/— | | —/Dig | | Bi/Dig | |
|---|---|---|---|---|---|---|---|---|---|---|
| | cut-off | individual | neg./pos. relative to buffer cut-off | | | | | | HPS cut-off | |
| HPS | 1044 | 2199 | 66 | 65 | 65 | 71 | 160 | 167 | 3026 | 2975 |
| | | 2198 | 53 | 53 | 51 | 56 | 63 | 75 | 828 | 862 |
| | | 2201 | 63 | 55 | 58 | 54 | 63 | 66 | 470 | 489 |
| | | 2195 | 55 | 54 | 53 | 55 | 103 | 100 | 2456 | 2515 |
| buffer | 144 | 2205 | 54 | 53 | 52 | 54 | 94 | 99 | 2452 | 2431 |
| | | 2202 | 53 | 56 | 57 | 54 | 123 | 169 | 3498 | 3598 |
| | | 2194 | 55 | 58 | 58 | 55 | 173 | 145 | 4690 | 4648 |
| | | 2196 | 64 | 69 | 54 | 67 | 72 | 81 | 856 | 882 |

The exchange of the substrate for the detection enzyme can reduce the signal-to-noise (S/N) ratio.
with detergent and HPPA (3-(4-hydroxyphenyl) propionic acid):

| | Bi/Dig | Bi/Dig | —/— | | Bi/— | | —/Dig | | Bi/Dig | |
|---|---|---|---|---|---|---|---|---|---|---|
| | cut-off | individual | neg./pos. relative to buffer cut-off | | | | | | HPS cut-off | |
| HPS | 1044 | 2199 | 66 | 65 | 65 | 71 | 160 | 167 | 3026 | 2975 |
| | | 2198 | 53 | 53 | 51 | 56 | 63 | 75 | 828 | 862 |
| | | 2201 | 63 | 55 | 58 | 54 | 63 | 66 | 470 | 489 |
| | | 2195 | 55 | 54 | 53 | 55 | 103 | 100 | 2456 | 2515 |
| buffer | 144 | 2205 | 54 | 53 | 52 | 54 | 94 | 99 | 2452 | 2431 |
| | | 2202 | 53 | 56 | 57 | 54 | 123 | 169 | 3498 | 3598 |
| | | 2194 | 55 | 58 | 58 | 55 | 173 | 145 | 4690 | 4648 |
| | | 2196 | 64 | 69 | 54 | 67 | 72 | 81 | 856 | 882 |

with detergent and TMB (3,3′,5,5′-tetramethyl-benzidine):

| | Bi/Dig | | Bi/Dig | | —/— | | Bi/— | | —/Dig | | Bi/Dig | |
|---|---|---|---|---|---|---|---|---|---|---|---|---|
| 50000 | 2.136 | 2.138 | 0.046 | 0.049 | 0.030 | 0.031 | 0.033 | 0.033 | 0.036 | 0.037 | 0.124 | 0.122 |
| 17857 | 1.126 | 1.119 | 0.043 | 0.047 | 0.029 | 0.029 | 0.029 | 0.029 | 0.032 | 0.031 | 0.048 | 0.050 |
| 6378 | 0.546 | 0.538 | 0.046 | 0.049 | 0.032 | 0.031 | 0.030 | 0.030 | 0.033 | 0.033 | 0.042 | 0.041 |
| 2278 | 0.313 | 0.310 | 0.045 | 0.048 | 0.028 | 0.030 | 0.030 | 0.029 | 0.033 | 0.033 | 0.100 | 0.099 |
| 813 | 0.162 | 0.162 | 0.029 | 0.033 | 0.028 | 0.028 | 0.029 | 0.027 | 0.033 | 0.032 | 0.092 | 0.092 |
| 291 | 0.084 | 0.092 | 0.029 | 0.033 | 0.029 | 0.030 | 0.030 | 0.026 | 0.035 | 0.035 | 0.127 | 0.127 |
| 104 | 0.068 | 0.060 | 0.028 | 0.032 | 0.028 | 0.029 | 0.030 | 0.028 | 0.034 | 0.035 | 0.154 | 0.158 |
| blank | 0.050 | 0.050 | 0.030 | 0.033 | 0.030 | 0.031 | 0.031 | 0.029 | 0.033 | 0.032 | 0.050 | 0.049 |

When using HPPA the cut-point (CP) was 4708 (approx. 817 ng/ml), whereas when using TMB the CP was 0.162 (approx. 845 ng/ml).

| | HPPA | | TMB | |
|---|---|---|---|---|
| | pool blank | S/N | pool blank | S/N |
| HPS | 1389 | 7.8 | 0.044 | 1.9 |
| buffer | 178 | | 0.023 | |

To avoid long-term incubation (over-night incubation) an acid dissociation step was introduced. The method then comprises a 30-minute incubation at pH 2, followed by pH adjustment to pH 7.4, addition of the biotinylated capture antibody and the digoxigenylated tracer antibody and a one-hour incubation.

Based on 8 individual samples an average value of 4410 fluorescence units with a coefficient of variation of 9% was determined. The cut-point was 5059 fluorescent units (1915 ng/ml). Thus, the introduction of the acid dissociation step did not result in an improvement in sensitivity.

The presence of plasma or serum did not significantly change the assay characteristics. Human and NHP serum provide for similar assay results (see FIG. 3).

Horse and rabbit serum provide similar results as buffer. Horse and rabbit C5 are not cross reactive. The blank signal of C5 depleted human serum is comparable to buffer and signals by dilution 4 are lower than with human pool serum (see FIG. 4).

The assay performance was impaired by the increase of horse serum and thereby horse C5 content in the sample (see FIG. 5).

Likewise, an addition of human C5 causes an increase in the signal confirming that the assay interference stems from the target of the therapeutic antibody in the sample (see FIG. 6).

It has been found that calibration of a bridging assay is possible with both, C5 and control pAb, i.e. C5 is causing signals in the assay (see FIG. 7). Without being bound by this theory it is assumed that sticky C5 aggregates are causing the problem (see FIG. 8 (wash buffer pH 7.4) and FIG. 9 (wash buffer pH 5.5)).

It has been found that C5 and pAb both bind to the therapeutic drug. Higher Drug to C5 ratio results in lower responses indicating no non-specific binding (see BIAcore data in FIG. 10).

It has been found that C5 and pAb both bind to the therapeutic drug using a therapeutic drug pre-coated surface. After capturing of C5 the surface is capable to bind therapeutic drug suggesting a that free epitopes are still present, which is the case for aggregated C5 (see FIG. 11).

Based on these findings the method has been further adapted by using an enhanced was step of washing 6 times with a buffer of a pH value of 5.5. Without being bound by this theory this reduces the amount/quantity of captured aggregates.
results in 1% serum; S/N=7; cut-point=2429 FU (0.82 ng/ml)

| individual | average OD | concentration |
|---|---|---|
| 2199 | 2005 | >blank < 104 ng/ml |
| 2198 | 1364 | >blank < 104 ng/ml |
| 2201 | 374 | <blank |
| 2195 | 1050 | >blank < 104 ng/ml |
| 2205 | 970 | >blank < 104 ng/ml |
| 2202 | 1881 | >blank < 104 ng/ml |
| 2194 | 1593 | >blank < 104 ng/ml |
| 2196 | 2188 | >blank < 104 ng/ml |

results in 0.1% serum; S/N=2; cut-point=2660 FU (0.84 ng/ml)

| individual | average OD | concentration |
|---|---|---|
| 2199 | 2670 | >blank < 104 ng/ml |
| 2198 | 1822 | >blank < 104 ng/ml |
| 2201 | 1187 | <blank |
| 2195 | 1507 | >blank < 104 ng/ml |
| 2205 | 1403 | >blank < 104 ng/ml |
| 2202 | 2148 | >blank < 104 ng/ml |
| 2194 | 1840 | >blank < 104 ng/ml |
| 2196 | 2262 | >blank < 104 ng/ml |

The S/N ratio lowers with dilution. The cut-point value and the lowest calibrator are similar in 1% and 0.1% serum containing samples. The cut-point was near 100 ng/ml for positive control in serum.

The assay was adapted to the findings as reported herein to reduce the formation of aggregates by performing an incubation at 4° C. incubation for about 16 hours (overnight), by the addition of 6.5 wt-% saccharose in assay buffer, and by the addition of the non-ionic detergent Brij 35.

The assay was further adopted by using a wash buffer with a pH value of 5.5 to reduce therapeutic drug to target, i.e. C5, interaction.

Based on the findings as outlined above a new assay format was established wherein the C5 aggregates present in the sample are removed prior to the determination and/or measurement and/or quantification of the anti-drug antibody in ta sample.

The new assay format as reported herein comprises a specific precipitation step wherein the target of the therapeutic antibody is precipitated at a pH value of about its pI value. In case of an anti-C5 antibody as therapeutic drug the target is human C5 and the precipitation is achieved by an incubation at a pH value in the range of 4.7 to 5.5. In one embodiment the incubation is at a pH value of about 5. In one preferred embodiment the incubation is at a pH value of about 5 for about 2 hours optionally with agitation.

The new assay format as reported herein comprises after the specific precipitation step an acid dissociation step. In this step, without being bound by this theory, the ADA (anti-anti-C5 antibody-antibody; anti-drug antibody) is dissociated from the precipitate. In one preferred embodiment the acid dissociation is by an incubation at a pH value of about 2 for about 5 minutes.

The new assay format as reported herein comprises optionally after the acid dissociation step a centrifugation step.

The new assay format as reported herein comprises after the acid dissociation step the step of adjusting the pH value of the sample to about 7.4, followed by the addition of the capture antibody and the tracer antibody with subsequent incubation. In one embodiment the capture antibody and the tracer antibody are the drug antibody. In one embodiment the capture antibody is conjugated to a first member of a binding pair. In one embodiment the binding pair is selected from biotin/(strept)avidin, hapten/anti-hapten antibody, nucleic acid/complementary nucleic acid, and ligand/ligand receptor. In one preferred embodiment the binding pair is biotin/(strept)avidin. In one preferred embodiment the capture antibody is conjugated to biotin. In one embodiment the tracer antibody is conjugated to a detectable label.

The new assay format as reported herein comprises after the incubation step with the capture and tracer antibody the immobilization of the capture antibody-anti-drug antibody-tracer antibody complex on a solid phase derivatized with the second member of the binding pair. In one preferred embodiment the second member of the binding pair is (strept)avidin.

The new assay format as reported herein comprises after the immobilization step the step of measuring and/or determining and/or quantifying the amount of the immobilized complex by incubating the immobilized complex with an antibody specifically binding to the detectable label conjugated to an enzyme catalyzing the conversion of a colorless substrate into a colored product followed by incubation with the colorless substrate of the enzyme, determination of the amount of formed colored product and correlating the amount of formed colored product with a calibration curve and thereby determining the amount of anti-drug antibody in the sample. In one embodiment the detectable label is a hapten. In one embodiment the hapten is selected from biotin, digoxigenin, theophylline and bromo desoxy uridine. In one preferred embodiment the detectable label is digoxygenin. In one preferred embodiment the enzyme is horseradish peroxidase. In one embodiment the colorless substrate is ABTS or HPPA or TMB. In one preferred embodiment the colorless substrate is TMB.

The results obtained with a method as reported herein (C5 precipitation approach) based on 30 individuals is presented in the following tables.
data of the calibration curve:

| concentration | average | STDEV | CV | calculated | recovery |
|---|---|---|---|---|---|
| [ng/ml] | [FU] | [FU] | [%] | [ng/ml] | [%] |
| 50000 | 41337 | 146 | 0.4 | 50054 | 100 |
| 17857 | 18883 | 151 | 0.8 | 19421 | 109 |
| 6378 | 7290 | 33 | 0.4 | 6107 | 96 |
| 2278 | 3010 | 91 | 3.0 | 2050 | 90 |
| 813 | 1475 | 16 | 1.1 | 827 | 102 |
| 291 | 774 | 9 | 1.2 | 347 | 119 |
| 104 | 340 | 4 | 1.2 | 96 | 93 |
| blank | 118 | 6 | 5.4 | | |

FU signals of the 30 individual samples:

| | | | |
|---|---|---|---|
| 102 | 103 | 116 | 132 |
| 133 | 98 | 96 | 97 |
| 105 | 100 | 99 | 113 |
| 112 | 112 | 101 | 102 |
| 114 | 122 | 115 | 109 |
| 116 | 123 | 130 | 112 |
| 136 | 131 | 133 | 116 |
| | 123 | | 133 |

| | |
|---|---|
| average | 114 |
| STDEV | 12.7 |
| CV | 11% |
| CP | 135 |

The new assay as reported herein has a suitable to high dynamic range, it is sensitive and addresses the interference from individual target levels present in the sample to be analyzed.

For the sample processing it is not required to remove the precipitate formed in the incubation step at about the pI of the target prior to the analysis. Without acid incubation step the interference is present (see FIG. 12):

A: with centrifugation after acid
treatment step (FIG. 12, 2)

| | |
|---|---|
| average | 114 |
| STDEV | 7.9 |
| CV | 7% |
| CP [FU] | 127 |
| CP [ng/ml] | 12 |

B: without centrifugation after
acid treatment step (FIG. 12, 1)

| | |
|---|---|
| average | 98 |
| STDEV | 9.3 |
| CV | 9% |
| CP [FU] | 113 |
| CP [ng/ml] | 17 |

C: no acid incubation step
(FIG. 12, 3)

| | |
|---|---|
| average | 3290 |
| STDEV | 4353 |
| CV | 132% |
| CP [FU] | 10428 |
| CP [ng/ml] | 2084 |

The calibration with positive control, long incubation time, and pH 5.5 wash is shown in FIG. 13. The average of 15 individuals is 119, the blank pool value is 123, and the cut-point is 135.

The calibration with polyclonal antibody, long incubation time, and pH 7.4 wash is shown in FIG. 14. The average of 15 individuals is 145, the blank pool value is 157, and the cut-point is 193.

The calibration with polyclonal antibody, short incubation time, and pH 5.5 wash is shown in FIG. 15. The average of 15 individuals is 124, the blank pool value is 115, and the cut-point is 247.

The calibration with polyclonal antibody in the presence of C5, 2-hour incubation at pH 5, and pH 5.5 wash is shown in FIG. 16.

The calibration with polyclonal antibody in the presence of C5, 2-hour incubation at pH 5, and pH 7.5 wash is shown in FIG. 17.

The calibration with polyclonal antibody in the presence of C5, 30-minute incubation at pH 2, and pH 5.5 wash is shown in FIG. 18 (ADA assay using acid dissociation according to the art).

### Exemplary Drug Antibody for Specific Embodiments of the Invention

US 2016/0167054 discloses anti-C5 antibodies and methods of using the same. In some embodiments, an isolated anti-C5 antibody disclosed binds to an epitope within the beta chain of C5 with a higher affinity at neutral pH than at acidic pH.

C5 is a 181 kDa protein found in normal serum at approximately 71 μg/ml (0.4 μM). C5 is glycosylated with about 1.5-3% of its mass attributed to carbohydrate. Mature C5 is a heterodimer of 106 kDa alpha chain that is disulfide linked to 66 kDa beta chain. C5 is synthesized as a single chain precursor protein (pro-C5 precursor) of 1577 amino acids (see, e.g., U.S. Pat. Nos. 6,355,245 and 7,432,356). The pro-C5 precursor is cleaved to yield the beta chain as an amino terminal fragment and the a chain as alpha carboxyl terminal fragment. The alpha chain and the beta chain polypeptide fragments are connected to each other via a disulfide bond and constitute the mature C5 protein.

Mature C5 is cleaved into the C5a and C5b fragments during activation of the complement pathways. C5a is cleaved from the alpha chain of C5 by C5 convertase as an amino terminal fragment comprising the first 65 amino acids of the alpha chain. The remaining portion of mature C5 is fragment C5b, which contains the rest of the alpha chain disulfide bonded to the beta chain. Approximately 20% of the 11 kDa mass of C5a is attributed to carbohydrate.

C5a is an anaphylatoxin. C5b combines with C6, C7, C8 and C9 to form the membrane attack complex (MAC, C5b-9, terminal complement complex (TCC)) at the surface of the target cell. When sufficient numbers of MACs are inserted into target cell membranes, MAC pores are formed to mediate rapid osmotic lysis of the target cells.

Anaphylatoxins can trigger mast cell degranulation, which releases histamine and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract granulocytes such as neutrophils, eosinophils, basophils and monocytes to the site of complement activation.

The activity of C5a is regulated by the plasma enzyme carboxypeptidase N that removes the carboxy-terminal arginine from C5a forming C5a-des-Arg derivative. C5a-des-Arg exhibits only 1% of the anaphylactic activity and polymorpho nuclear chemotactic activity of unmodified C5a.

While a properly functioning complement system provides a robust defense against infecting microbes, inappropriate regulation or activation of complement has been implicated in the pathogenesis of a variety of disorders including, e.g., rheumatoid arthritis (RA); lupus nephritis; ischemia-reperfusion injury; paroxysmal nocturnal hemoglobinuria (PNH); atypical hemolytic uremic syndrome (aHUS); dense deposit disease (DDD); macular degeneration (e.g., age-related macular degeneration (AMD)); hemolysis, elevated liver enzymes, and low platelets (HELLP) syndrome; thrombotic thrombocytopenia purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis (see, e.g., Holers et al., Immunol. Rev. 223 (2008) 300-316). Therefore, inhibition of excessive or uncontrolled activations of the complement cascade can provide clinical benefits to patients with such disorders, especially to patients with Paroxysmal nocturnal hemoglobinuria (PNH).

Eculizumab is a humanized monoclonal antibody directed against the complement protein C5, and the first therapy approved for the treatment of paroxysmal nocturnal hemoglobinuria (PNH) and atypical hemolytic uremic syndrome (aHUS) (see, e.g., Dmytrijuk et al., The Oncologist 13 (2008) 894-910). Eculizumab inhibits the cleavage of C5 into C5a and C5b by C5 convertase, which prevents the generation of the terminal complement complex C5b-9. Both C5a and C5b-9 cause the terminal complement-mediated events that are characteristic of PNH and aHUS (see also, WO 2005/065607, WO 2007/96586, WO 2008/060790, and WO 2010/054403). Several reports have described other anti-C5 antibodies. For example, WO 86/28707 described an anti-C5 antibody which binds to the alpha chain of C5 but does not bind to C5a, and blocks the activation of C5, while WO 2002/30886 described an anti-C5 monoclonal antibody which inhibits C5a formation. On the other hand, WO 2004/006653 described an anti-C5 antibody which recognizes the proteolytic site for C5 convertase on the alpha chain of C5, and inhibits the conversion of C5 to C5a and C5b. WO 2010/015608 described an anti-C5 antibody which has an affinity constant of at least 1×10E7 M−1. In one embodiment the drug is Eculizumab.

In some embodiments, the method is for the detection of an ADAs against an anti-C5 antibody binding to an epitope within the beta chain of C5. In some embodiments, the anti-C5 antibody binds to an epitope within the MG1-MG2 domain of the beta chain of C5. In some embodiments, the anti-C5 antibody binds to an epitope within a fragment consisting of amino acids 27-115 of the beta chain (SEQ ID NO: 31) of C5. In some embodiments, the anti-C5 antibody binds to an epitope within the beta chain (SEQ ID NO: 31) of C5 which comprises at least one fragment selected from the group consisting of amino acids 38-48, 61-67, and 98-101. In some embodiments, the anti-C5 antibody binds to an epitope within a fragment of the beta chain (SEQ ID NO: 31) of C5 which comprises at least one amino acid residue selected from the group consisting of Glu48, Asp51, His61, His63, Lys100, and His101 of SEQ ID NO: 31. In further embodiments, the antibody binds to C5 with a higher affinity at neutral pH than at acidic pH. In further embodiments, the antibody binds to C5 with a higher affinity at pH 7.4 than at pH 5.8. In another embodiment, the anti-C5 antibody binds to the same epitope as an antibody described in Table 1. In further embodiments, the antibody binds to the same epitope as an antibody described in Table 1 with a higher affinity at pH 7.4 than at pH 5.8. In a further embodiment, the anti-C5 antibody binds to the same epitope as an antibody described in Tables 2 or 3. In further embodiments, the antibody binds to the same epitope as an antibody described in Tables 2 or 3 with a higher affinity at pH 7.4 than at pH 5.8.

TABLE 1
SEQ ID NO:

| | | | HVR- | HVR- | HVR- | HVR- | HVR- | HVR- |
|---|---|---|---|---|---|---|---|---|
| antibody | VH | VL | H1 | H2 | H3 | L1 | L2 | L3 |
| CFA0305 | 1 | 11 | 36 | 46 | 56 | 66 | 76 | 86 |
| CFA0307 | 2 | 12 | 37 | 47 | 57 | 67 | 77 | 87 |
| CFA0357 | 3 | 13 | 38 | 48 | 58 | 68 | 78 | 88 |
| CFA0501 | 4 | 14 | 39 | 49 | 59 | 69 | 79 | 89 |
| CFA0538 | 5 | 15 | 40 | 50 | 60 | 70 | 80 | 90 |
| CFA0590 | 6 | 16 | 41 | 51 | 61 | 71 | 81 | 91 |
| CFA0567 | 7 | 17 | 42 | 52 | 62 | 72 | 82 | 92 |
| CFA0573 | 8 | 18 | 43 | 53 | 63 | 73 | 83 | 93 |
| CFA0576 | 9 | 19 | 44 | 54 | 64 | 74 | 84 | 94 |

TABLE 2
SEQ ID NO:

| antibody | VH | HVR-H1 | HVR-H2 | HVR-H3 |
|---|---|---|---|---|
| 305L05 | 10 | 45 | 55 | 65 |
| 305L015 | 97 | 108 | 109 | 112 |
| 305L016 | 98 | 108 | 110 | 112 |
| 305L018 | 99 | 108 | 109 | 112 |
| 305L019 | 100 | 108 | 109 | 112 |
| 305L020 | 100 | 108 | 109 | 112 |
| 305L022 | 100 | 108 | 109 | 112 |
| 305L023 | 101 | 108 | 111 | 112 |

TABLE 3
SEQ ID NO:

| antibody | VL | HVR-L1 | HVR-L2 | HVR-L3 |
|---|---|---|---|---|
| 305L05 | 20 | 75 | 85 | 95 |
| 305L015 | 102 | 113 | 114 | 116 |
| 305L016 | 102 | 113 | 114 | 116 |
| 305L018 | 102 | 113 | 114 | 116 |
| 305L019 | 102 | 113 | 114 | 116 |
| 305L020 | 103 | 113 | 114 | 116 |
| 305L022 | 104 | 113 | 115 | 116 |
| 305L023 | 104 | 113 | 115 | 116 |

In certain embodiments, the anti-C5 antibody competes for binding to C5 with an antibody comprising a VH and VL pair selected from: (a) a VH of SEQ ID NO: 01 and a VL of SEQ ID NO: 11; (b) a VH of SEQ ID NO: 05 and a VL of SEQ ID NO: 15; (c) a VH of SEQ ID NO: 04 and a VL of SEQ ID NO: 14; (d) a VH of SEQ ID NO: 06 and a VL of SEQ ID NO: 16; (e) a VH of SEQ ID NO: 02 and a VL of SEQ ID NO: 12; (f) a VH of SEQ ID NO: 03 and a VL of SEQ ID NO: 13; (g) a VH of SEQ ID NO: 09 and a VL of SEQ ID NO: 19; (h) a VH of SEQ ID NO: 07 and a VL of SEQ ID NO: 17; (i) a VH of SEQ ID NO: 08 and a VL of SEQ ID NO: 18; and (j) a VH of SEQ ID NO: 10 and a VL of SEQ ID NO: 20.

In certain embodiments, the anti-C5 antibody is for use as a medicament. In one embodiment the anti-C5 antibody is used in treating a complement-mediated disease or condition which involves excessive or uncontrolled activation of C5. In additional embodiments, the anti-C5 antibody is used in treating diseases or disorders that include but are not limited to, paroxysmal nocturnal hemoglobinuria (PNH), age-related macular degeneration, myocardial infarction, rheumatoid arthritis, osteoporosis, osteoarthritis, and inflammation. The anti-C5 antibody is used to enhance the clearance of C5 from plasma.

In certain embodiments, the method is for the detection of ADAs against an anti-C5 antibody comprising a VH as in any of the embodiments provided above and a heavy chain constant region comprising the amino acid sequence of any one of SEQ ID NOs: 27, 28, 29, 105, 106, and 107. In certain embodiments, the method is for the detection of an anti-C5 antibody comprising a VL as in any of the embodiments provided above and a light chain constant region comprising the amino acid sequence of any one of SEQ ID NOs: 36, 37, and 38.

In certain embodiments, the method is for the detection of ADAs against an anti-C5 antibody that competes for binding to C5 with an antibody comprising a VH and VL pair selected from: (a) a VH of SEQ ID NO: 01 and a VL of SEQ ID NO: 11; (b) a VH of SEQ ID NO: 22 and a VL of SEQ ID NO: 25; (c) a VH of SEQ ID NO: 21 and a VL of SEQ ID NO: 24; (d) a VH of SEQ ID NO: 05 and a VL of SEQ ID NO: 15; (e) a VH of SEQ ID NO: 04 and a VL of SEQ ID NO: 14; (f) a VH of SEQ ID NO: 06 and a VL of SEQ ID NO: 16; (g) a VH of SEQ ID NO: 02 and a VL of SEQ ID NO: 12; (h) a VH of SEQ ID NO: 03 and a VL of SEQ ID NO: 13; (i) a VH of SEQ ID NO: 09 and a VL of SEQ ID NO: 19; (j) a VH of SEQ ID NO: 7 and a VL of SEQ ID NO: 17; (k) a VH of SEQ ID NO: 8 and a VL of SEQ ID NO: 18; (l) a VH of SEQ ID NO: 23 and a VL of SEQ ID NO: 26; and (m) a VH of SEQ ID NO: 10 and a VL of SEQ ID NO: 20.

In certain embodiments, the method is for the detection of ADAs against an anti-C5 antibody that competes for binding C5 with an antibody comprising a VH and VL pair selected from: (a) a VH of SEQ ID NO: 22 and a VL of SEQ ID NO: 25; (b) a VH of SEQ ID NO: 21 and a VL of SEQ ID NO: 24; (c) a VH of SEQ ID NO: 05 and a VL of SEQ ID NO: 15; (d) a VH of SEQ ID NO: 04 and a VL of SEQ ID NO: 14; (e) a VH of SEQ ID NO: 06 and a VL of SEQ ID NO: 16; (f) a VH of SEQ ID NO: 02 and a VL of SEQ ID NO: 12; (g) a VH of SEQ ID NO: 03 and a VL of SEQ ID NO: 13; (h) a VH of SEQ ID NO: 09 and a VL of SEQ ID NO: 19; (i) a VH of SEQ ID NO: 07 and a VL of SEQ ID NO: 17; (j) a VH of SEQ ID NO: 8 and a VL of SEQ ID NO: 18; (k) a VH of SEQ ID NO: 23 and a VL of SEQ ID NO: 26.

The following Examples, Sequences and Figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1: Effect of different capture antibody as well as tracer antibody concentration ((1): 500 ng/ml each, (2): 1000 ng/ml each, (3): 1500 ng/ml each, (4): 2000 ng/ml each).

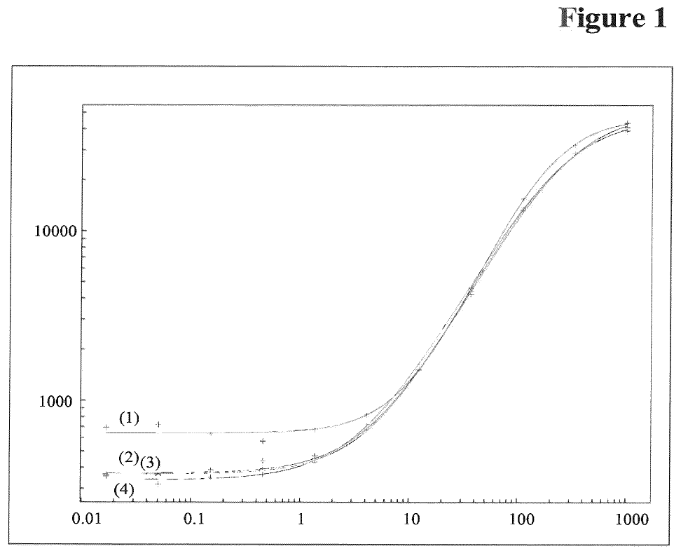

FIG. 2: Effect of different peroxidase concentrations ((1): 5 mU, (2): 10 mU, (3): 25 mU, (4): 50 mU at 1000 ng/ml capture and tracer antibody concentration).

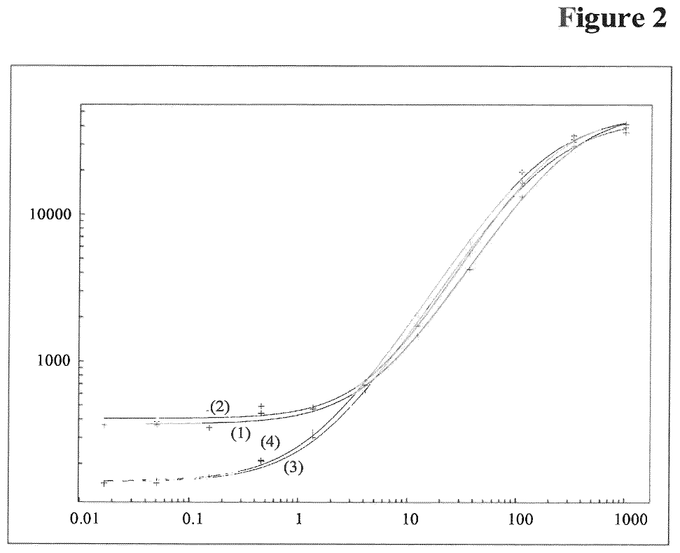

FIG. 3: Effect of Human and NHP serum ((1): human serum, (2): human plasma, (3): cynomolgus serum).

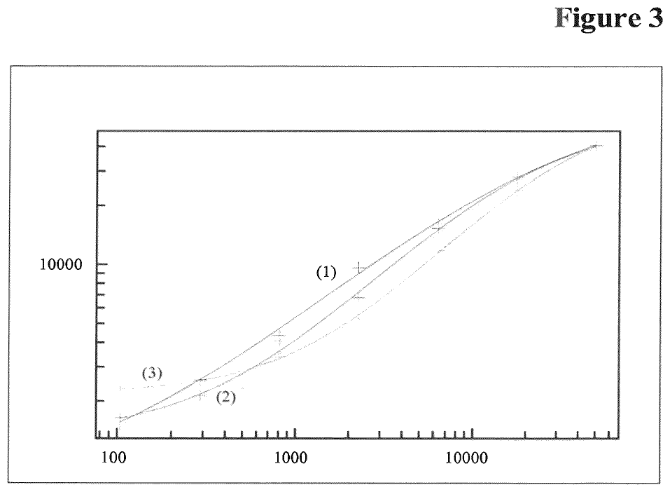

FIG. 4: Effect of horse (1), rabbit (2), C5 depleted human plasma (3) and buffer (4).

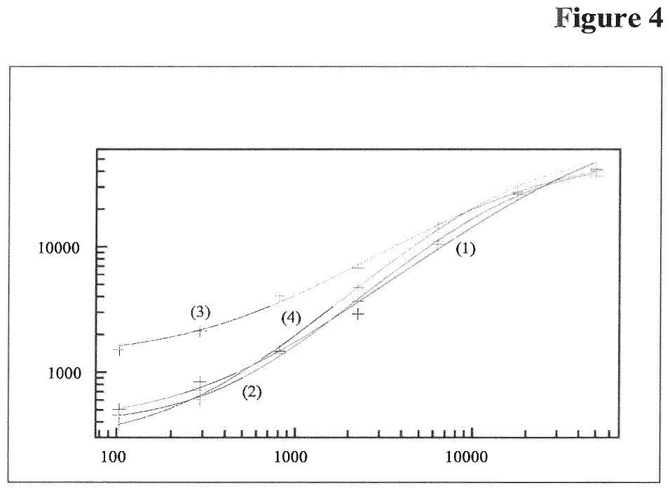

FIG. 5: The assay performance was impaired by the increase of horse serum and thereby horse C5 content in the sample (see FIG. 5) (1% human serum in 0% (1), 1% (2), 5% (3) or 10% (4) horse serum).

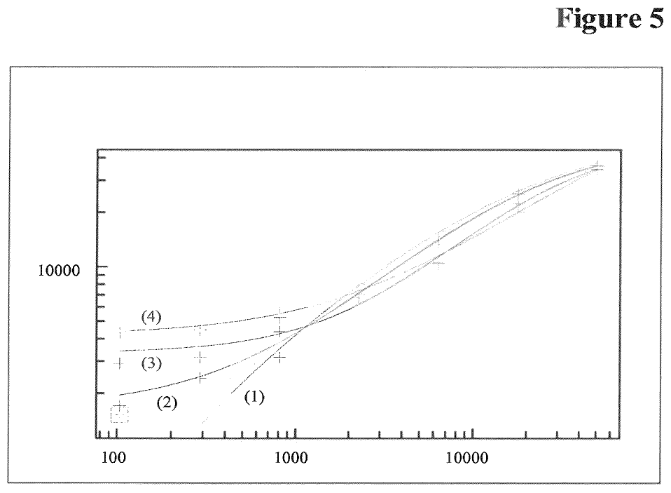

FIG. 6: Effect of human C5 on the assay (pAb+ (1): human serum, (2): buffer+brij, (3): 500 ng/ml C5 in human serum, (4): 500 ng/ml C5 in buffer+brij).

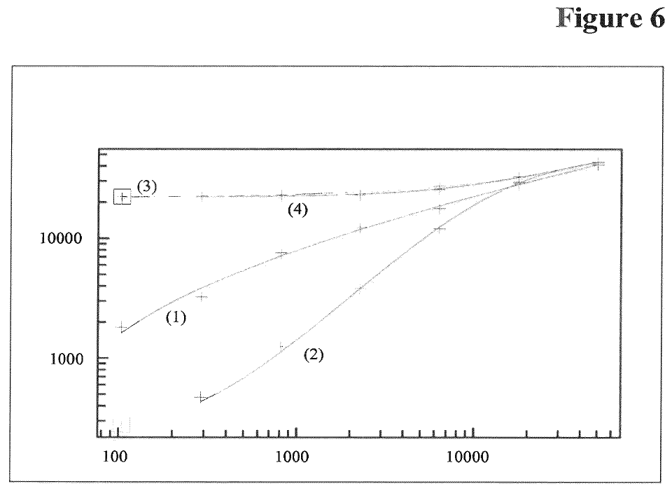

FIG. 7: Calibration of a bridging assay with C5 ((1): pH 7.4, (2): pH 5.5, (3): pH 8.0) and control pAb (anti-idiotypic antibody) ((4): pH 7.4, (5): pH 5.5, (6): pH 8.0).

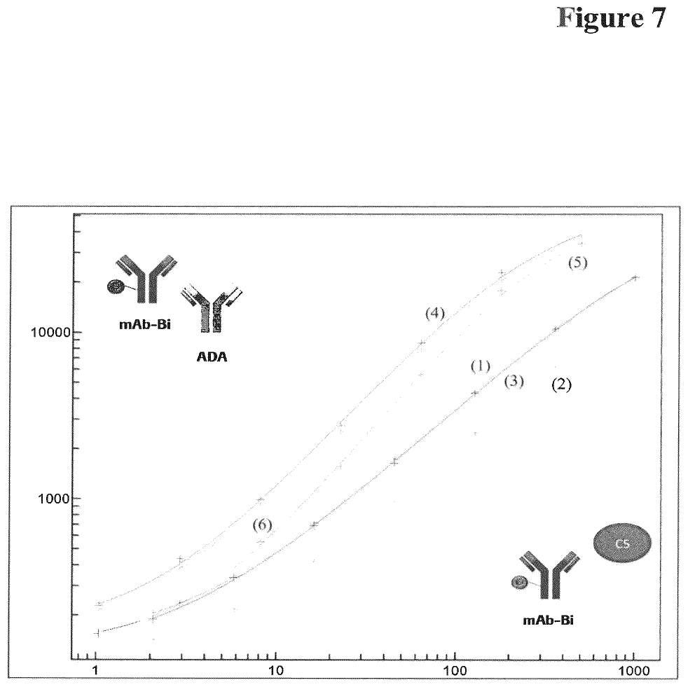

FIG. 8: Use of wash buffer pH 7.4 in the bridging assay (calibration with (1) pAb anti-idiotypic antibody and capture and tracer antibody, (2) C5 with no capture and no tracer antibody, (3) C5 with capture and no tracer antibody, (4) C5 with no capture but with tracer antibody, (5) C5 with capture and tracer antibody).

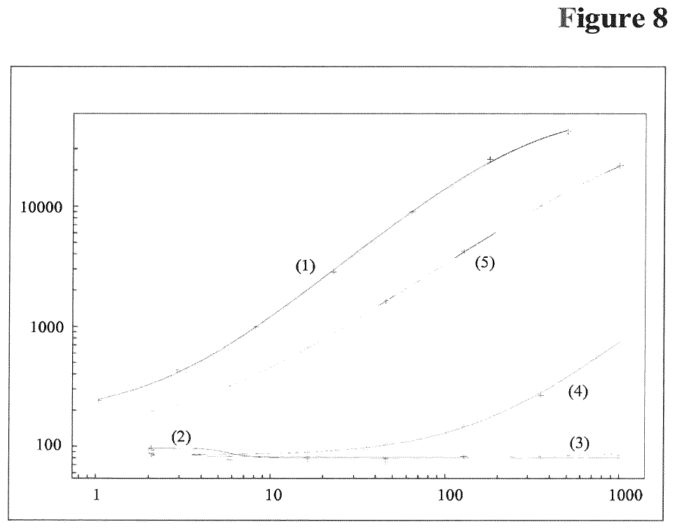

FIG. 9: Use of wash buffer pH 5.5 in the bridging assay (calibration with (1) pAb anti-idiotypic antibody and capture and tracer antibody, (2) C5 with no capture and no tracer antibody, (3) C5 with capture and no tracer antibody, (4) C5 with no capture but with tracer antibody, (5) C5 with capture and tracer antibody).

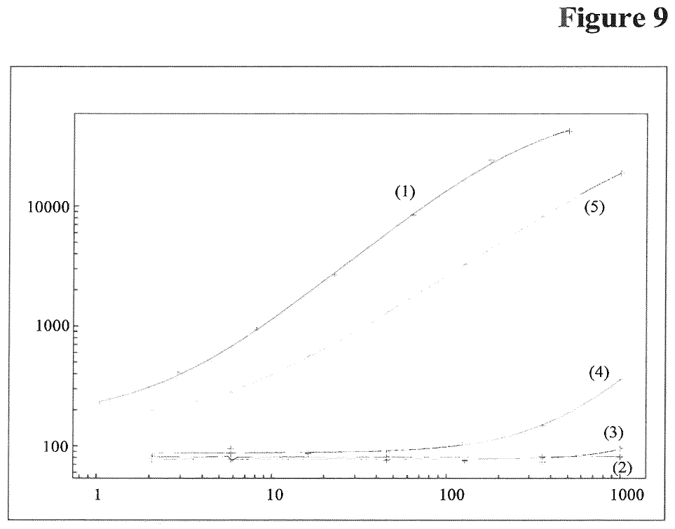

FIG. 10: Effect of drug to C5 ratio; adjusted SPR sensogram; (1): C5, (2): C5+pAb 100/100 nM, (3): C5+pAb 100/10 nM, (4): C5+pAb 100/250 nM, (5): C5+pAb 100/500 nM, (6): C5+pAb 100/50 nM, (7): mAb-C5, (8): pAb-anti-idiotypic-mAb C5, (9) buffer.

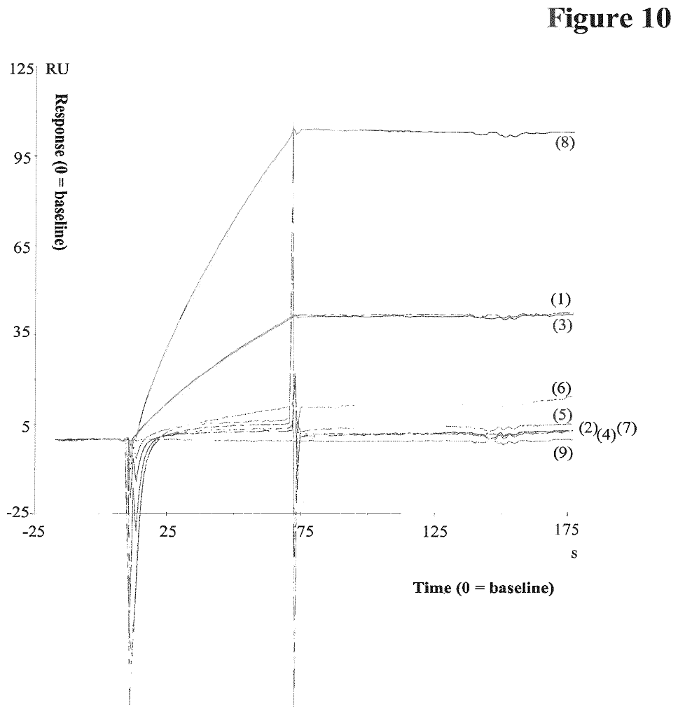

FIG. 11: Binding of pAb (green) and C5 (red) to a biotinylated drug coated surface followed by addition at time as indicated by arrow of buffer or tracer (1, 2)/digoxigenylated drug (3, 4).

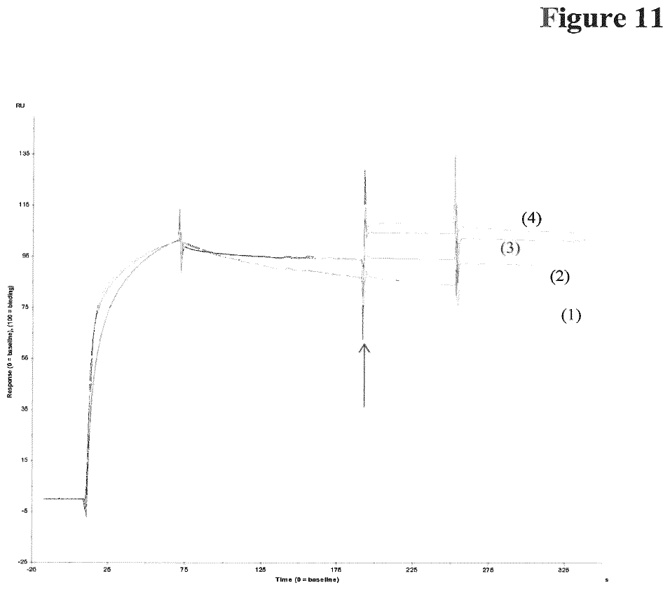

FIG. 12: Calibration of pAb in the bridging assay (1) without centrifugation (2) with centrifugation compared to a calibration without applying the acid incubation step (3).

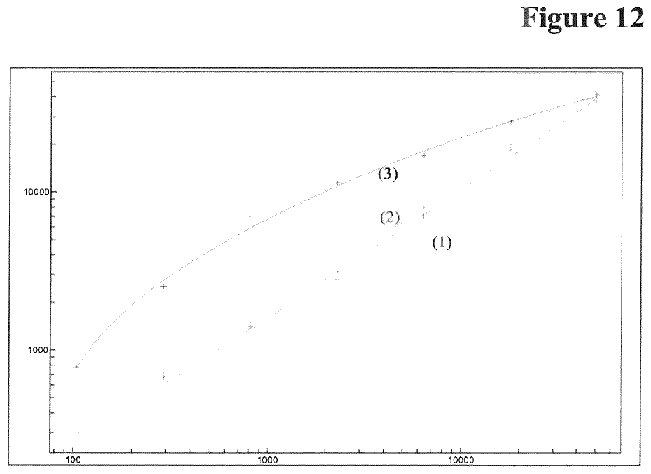

FIG. 13: Calibration with positive control, long incubation time, and pH 5.5 wash.

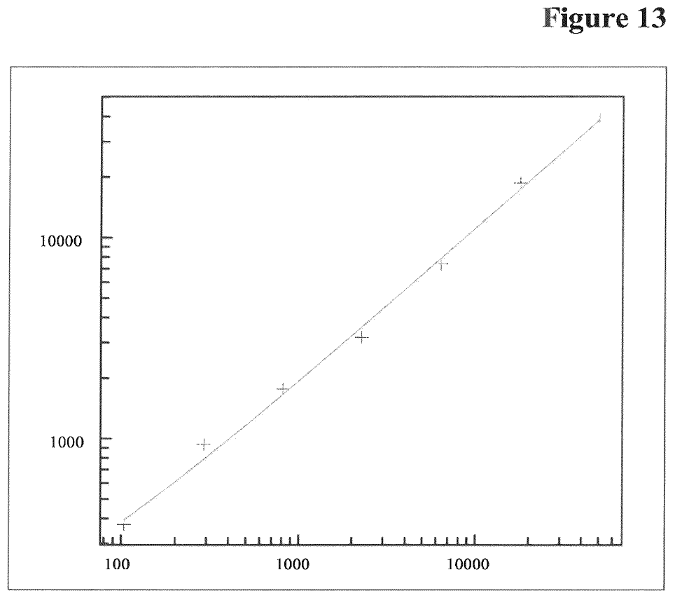

FIG. 14: Calibration with polyclonal antibody, long incubation time, and pH 7.4 wash.

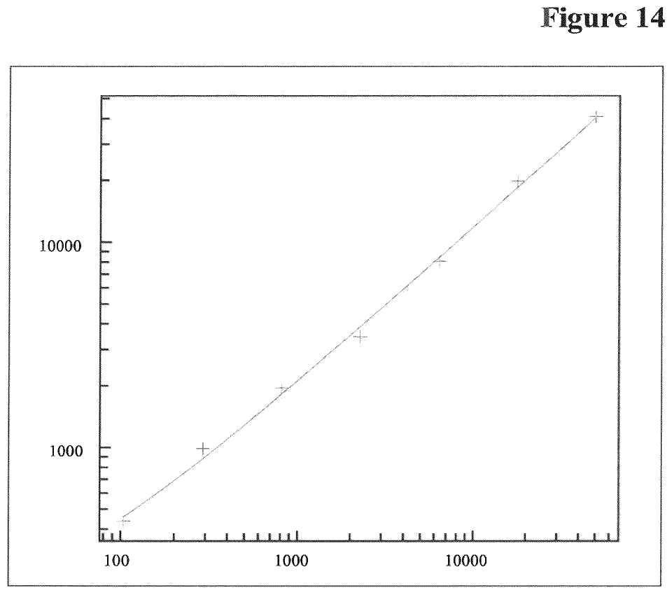

FIG. 15: Calibration with polyclonal antibody, short incubation time, and pH 5.5 Confidential wash.

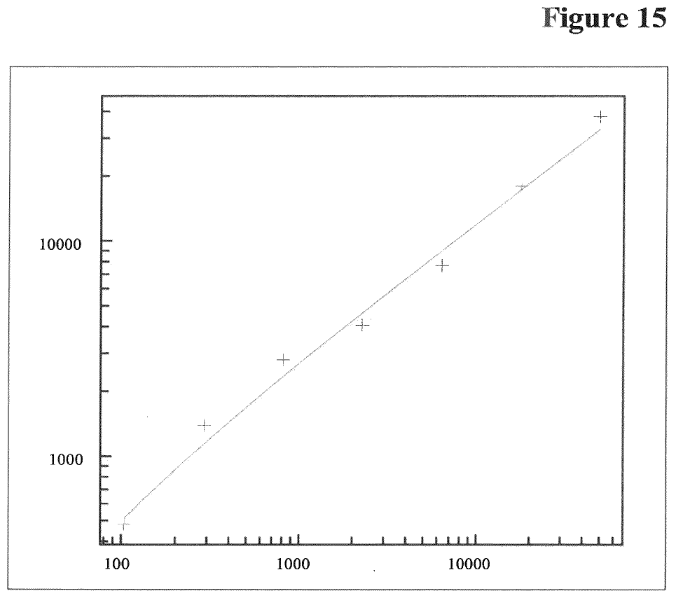

FIG. 16: Calibration with polyclonal antibody in the presence of serum (1), buffer (2), 2-hour incubation at pH 5, and pH 5.5 wash, calibration with C5 in the presence of serum (3), buffer (4), 2-hour incubation at pH 5, and pH 5.5 wash.

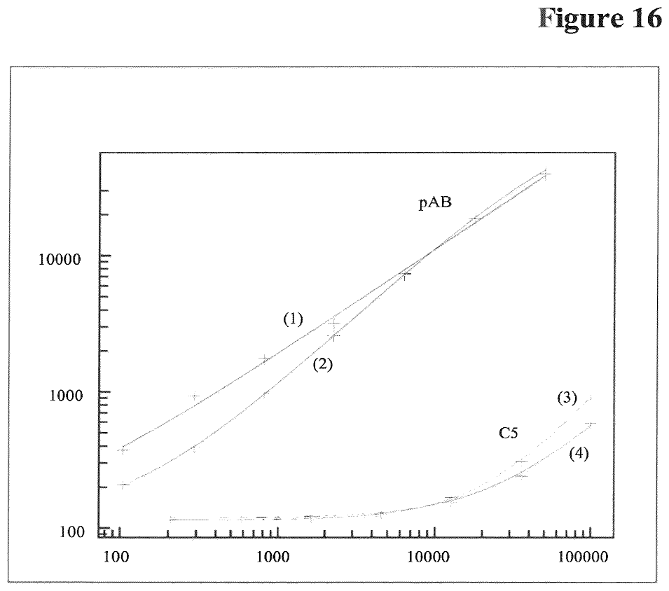

FIG. 17: Calibration with polyclonal antibody in the presence of serum (1), buffer (2), 2-hour incubation at pH 5, and pH 7.4 wash, calibration with C5 in the presence of serum (3), buffer (4), 2-hour incubation at pH 5, and pH 7.4 wash.

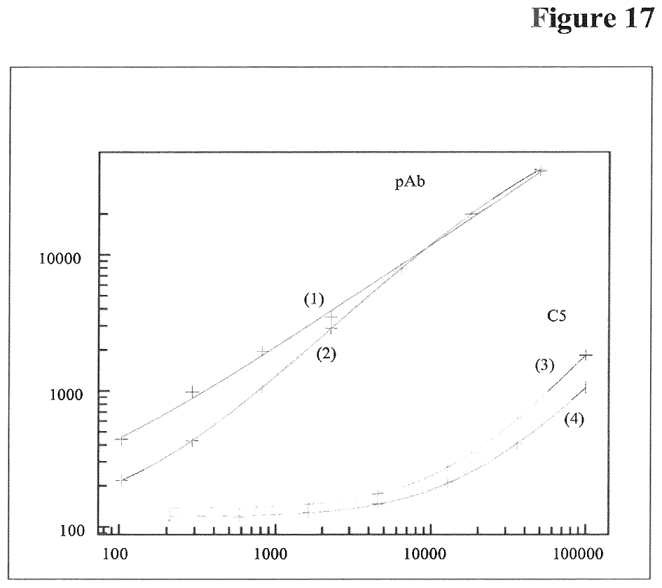

FIG. 18: Calibration with polyclonal antibody in the presence of serum (1), buffer (2), 0.5-hour incubation at pH 2, and pH 5.5 wash, calibration with C5 in the presence of serum (3), buffer (4), 0.5-hour incubation at pH 2, and pH 5.5 wash.

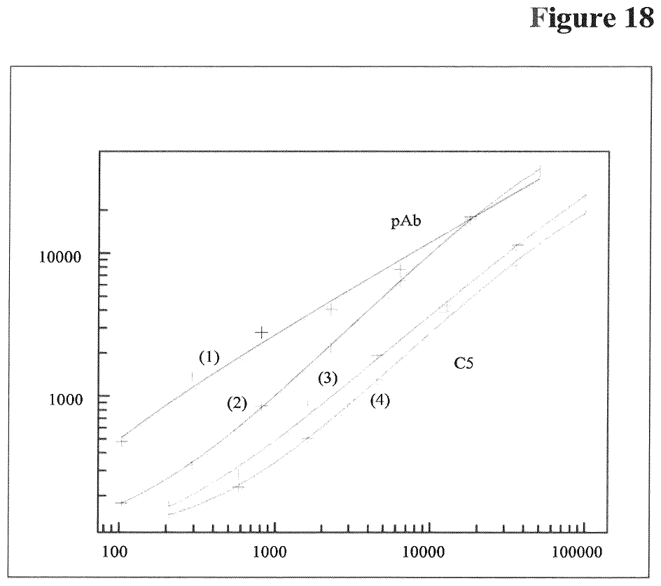

### EXAMPLES

### Example 1

Assay with Saccharose and Brij

Biotinylated and digoxigenylated drug was incubated with 30 individual sera samples. For functionally testing of the used reagents (control) serum samples (pooled serum) were prepared with different concentrations of an artificial positive control standard, incubated and processed as the individual serum samples. The labelled drug concentrations were kept constant at 1000 ng/ml each. The final serum concertation in the assay was 1%. Formed immune complexes were transferred to a white Streptavidin (SA)-coated microtiter plate and incubated for 1 hour to immobilize the complexes via the biotin-labeled capture reagent. Following aspiration of the supernatant unbound substances were removed by repeated washings. Immobilized complexes were incubated with an anti-digoxigenin antibody conjugated to horseradish peroxidase (anti-digoxigenin-POD (poly)). Each step was performed with the same buffer by using either a PBS buffer with 6.5% saccharose or Roche universal buffer for ELISA with Brij 35 at a concentration of 0.5%. Finally, formed immobilized immune complexes were visualized by addition of oxidized HPPA solution, a fluorescent POD substrate. The emission was photometrically determined (Excitation at 320 nm, emission at 405 nm wave length) and set in relation to the positive control concentration in the sample. The CV of the individual serum samples are 29% (saccharose buffer assay) and 181% (Roche universal buffer with Brij).

### Example 2

Assay with Acid Incubation Step

Individual serum samples (N=30) and artificial positive control samples were incubated for 2 hours with 10 mM acetate buffer pH 5.0. Thereafter the samples were incubated for 5 min with 0.1 M glycine hydrochloride pH 2.0. The acidified samples were mixed with biotinylated capture antibody and digoxigenylated detection antibody, neutralized with 0.5 M TRIS buffer pH 8.5 and incubated for 30 min at RT and 450 rpm on a microtiter plate shaker. The final serum assay concentration was 1%. Formed immune complexes were transferred to a Streptavidin (SA)-coated microtiter plate and incubated for 1 hour to immobilize the immune complexes via the biotin-labeled capture antibody. Following aspiration of the supernatant unbound substances were removed by repeated washings. Immobilized immune complexes were incubated with an anti-digoxigenin Fab fragment conjugated to horseradish peroxidase (Anti-Dig-POD). Formed immobilized immune complexes were visualized by addition of oxidized HPPA solution, a fluorescent POD substrate. The emission was photometrically determined (Excitation at 320 nm, emission at 405 nm wave length) and set in relation to the artificial positive control concentration in the serum sample. The artificial positive control provides a blank to noise ratio at 100 ng/ml in 100% serum of >3. The CV of the individual serum samples (N=30) is 7%.

### Example 3

Assay with Low Serum Contend and No Acid Dissociation

Biotinylated and digoxigenylated drug was incubated with 32 individual sera at a final serum concentration of 1% and 0.1%. For functionally testing of the used reagents (control) serum samples (pooled serum) were prepared with different concentrations of an artificial positive control standard, incubated and processed as the individual serum samples. The labelled drug concentrations were kept constant at 1000 ng/ml each. Formed immune complexes were transferred to a white Streptavidin (SA)-coated microtiter plate and incubated for 1 hour to immobilize the complexes via the biotin-labeled capture reagent. Following aspiration of the supernatant unbound substances were removed by repeated washings. Immobilized complexes were incubated with an anti-digoxigenin antibody conjugated to horseradish peroxidase (anti-digoxigenin-POD (poly)). Finally, formed immobilized immune complexes were visualized by addition of oxidized HPPA solution, a fluorescent POD substrate. The emission was photometrically determined (Excitation at 320 nm, emission at 405 nm wave length) and was proportional to the positive control concentration in the sample. The artificial positive control indicates an assay sensitivity of ˜100 ng/ml in 100% serum for the 1 and 0.1% assay. The CV of the individual serum samples are 74% (0.1% serum assay) and 65% (1% serum assay).

1. An immunoassay for quantifying the amount of anti-drug antibody, which anti-drug antibody can specifically bind to a drug antibody, which drug antibody can specifically bind to a therapeutic target, in a serum or plasma sample with reduced target interference comprising the following steps:
a) incubating an immobilized capture antibody with a serum or plasma sample comprising drug, target and anti-drug antibody, to form a capture antibody-anti-drug antibody complex,
b) washing the complex formed in step a) with a buffer comprising a sugar and a detergent, which has a pH value of about the pI of the target,
c) incubating for 12 to 24 hours the washed complex of step b) with a tracer antibody conjugated to a label to form a capture antibody-anti-drug antibody-tracer antibody complex,
d) quantifying the amount of anti-drug antibody by determining the label in the complex formed in step c).

2. The immunoassay according to claim 1, wherein the tracer antibody and the capture antibody is the drug antibody.

3. The immunoassay according to any one of claims 1 to 2, wherein the immunoassay comprises a capture antibody, a tracer antibody and a detection antibody, wherein the capture antibody is the drug conjugated to a first member of a binding pair, the tracer antibody is the drug antibody conjugated to a detectable label and the detection antibody is an antibody specifically binding to the detectable label conjugated to an enzyme.

4. The immunoassay according to any one of claims 1 to 3, wherein the capture antibody and/or the tracer antibody is independently of each other selected from the group consisting of complete/full length drug antibody, F(ab′)2, Fab and scFv.

5. The immunoassay according to any one of claims 1 to 4, wherein the sugar is a monosaccharide, a disaccharide or a trisaccharide.

6. The immunoassay according to any one of claims 1 to 5, wherein the sugar is selected from the group of disaccharides consisting of saccharose, lactose, maltose, iso-maltose, and trehalose.

7. The immunoassay according to any one of claims 1 to 6, wherein the sugar has a concentration of about 6.5 wt-%.

8. The immunoassay according to any one of claims 1 to 7, wherein the detergent is a non-ionic detergent.

9. The immunoassay according to any one of claims 1 to 8, wherein the detergent is selected from the group of detergents consisting of polyalkylene glycol ether (trade name Brij), polyoxyethylene sorbitane monoesters (trade name Tween), octylphenol ethoxylate (trade name Trion or Nonident), octyl-beta-glycoside, n-fatty acid-N-methyl-D-glucamide (trade name MEGA), and N,N′-bis-(3-D-gluconamidopropyl) cholamide (tradename CHAP).

10. The immunoassay according to any one of claims 1 to 9, the incubation is for 14 to 20 hours. In one embodiment the incubating is for 15 to 17 hours.

11. The immunoassay according to any one of claims 3 to 10, wherein the wherein the first member of the binding pair is selected from the group consisting of hapten, antigen and hormone.

12. The immunoassay according to any one of claims 1 to 11, wherein the binding pair is selected from the group consisting of biotin/(strept)avidin, theophylline/anti-theophylline antibody, 5-bromo-desoxy-uridine/anti-5-bromo-deoxy-uridine antibody, digoxigenin/anti-digoxygenin antibody, and helicar/anti-helical antibody.

13. The immunoassay according to any one of claims 1 to 12, wherein the drug is an anti-C5 antibody and the target is human C5.

14. The immunoassay according to any one of claims 1 to 13, wherein the sugar is saccharose, the detergent is polyethylene glycol dodecyl ether, the drug is an anti-C5 antibody, the target is human C5 and the buffer has a pH value of about 5.5.

15. An immunoassay for quantifying the amount of anti-drug antibody, which anti-drug antibody can specifically bind to a drug antibody, which drug antibody can specifically bind to a therapeutic target, in a serum or plasma sample with reduced target interference comprising the following steps:
a) incubating the serum or plasma sample at a pH value that is about the pI value of the target, and optionally removing formed precipitate after the incubation,
b) incubating the serum or plasma sample obtained in step a) at a pH value of about 2, and optionally centrifuging the incubated sample to remove formed precipitate,
c) adjusting the pH value to about 7.4, adding capture antibody conjugated to a first member of a binding pair and tracer antibody conjugated to a detectable label to the serum or plasma sample obtained in step b) and incubating the mixture to form a capture antibody-anti-drug antibody-tracer antibody-complex,
d) quantifying the complex formed in step c) and thereby quantifying the amount of anti-drug antibody in the serum or plasma sample.

16. The immunoassay according to claim 15, wherein the step of quantifying the capture antibody-anti-drug antibody-tracer antibody-complex (step d)) comprises the steps of
d1) incubating the serum or plasma sample obtained in step c) with the second member of the binding pair conjugated to a solid surface to capture the capture antibody-anti-drug antibody-tracer antibody-complex, and optionally washing the surface,
d2) quantifying the amount of anti-drug antibody by determining the detectable label in the complex formed in step d1).

17. The immunoassay according to any one of claims 15 to 16, wherein the incubation at about the pI value of the target is at a pH value in the range of 0.5 pH units below the pI of the target to 0.5 pH units above the pI value of the target.

18. The immunoassay according to any one of claims 15 to 17, wherein the incubating in step a) is with agitation.

19. The immunoassay according to any one of claims 15 to 18, wherein the incubating in step a) is for 1.5 to 2.5 hours.

20. The immunoassay according to any one of claims 15 to 19, wherein the incubating in step b) is for about 5 min.

21. The immunoassay according to any one of claims 15 to 20, wherein the incubating in stop d) is for about 60 min.

22. The immunoassay according to any one of claims 15 to 21, wherein the tracer antibody and the capture antibody is the drug antibody.

23. The immunoassay according to any one of claims 15 to 22, wherein the immunoassay comprises a capture antibody, a tracer antibody and a detection antibody, wherein the capture antibody is the drug conjugated to a first member of a binding pair, the tracer antibody is the drug antibody conjugated to a detectable label and the detection antibody is an antibody specifically binding to the detectable label conjugated to an enzyme.

24. The immunoassay according to any one of claims 15 to 23, wherein the capture antibody and/or the tracer antibody is independently of each other selected from the group consisting of complete/full length drug antibody, F(ab′)2, Fab and scFv.

25. The immunoassay according to any one of claims 15 to 24, wherein the first member of the binding pair is selected from the group consisting of hapten, antigen and hormone.

26. The immunoassay according to any one of claims 15 to 25, wherein the binding pair is selected from the group consisting of biotin/(strept)avidin, theophylline/anti-theophylline antibody, 5-bromo-desoxy-uridine/anti-5-bromo-deoxy-uridine antibody, digoxigenin/anti-digoxygenin antibody, and helicar/anti-helical antibody.

27. The immunoassay according to any one of claims 15 to 26, wherein the drug is an anti-C5 antibody and the target is human C5. In one embodiment the pH value in step a) is in the range of pH 4.7 to pH 5.5.

28. The immunoassay according to any one of claims 15 to 27, wherein the immunoassay for quantifying the amount of anti-drug antibody, which anti-drug antibody can specifically bind to a drug antibody, which drug antibody is an anti-C5 antibody that can specifically bind to human C5, in a serum or plasma sample comprises the following steps:
a) incubating the serum or plasma sample at a pH value in the range of 4.7 to 5.5 for 1.5 to 2.5 hours, and optionally removing formed precipitate after the incubation,
b) incubating the scrum or plasma sample obtained in step a) at a pH value of about 2 for about 5 minutes, and optionally centrifuging the incubated sample to remove formed precipitate,
c) adjusting the pH value to about 7.4, adding capture drug antibody conjugated to biotin and tracer drug antibody conjugated to digoxigenin to the serum or plasma sample obtained in step b) and incubating the mixture to form a capture antibody-anti-drug antibody-tracer antibody-complex,
d) incubating the serum or plasma sample obtained in step c) with (strept)avidin conjugated to a solid surface to capture the capture antibody-anti-drug antibody-tracer antibody-complex, and optionally washing the surface,
e) detecting anti-drug antibody by determining the digoxigenin in the complex formed in step d) by incubating with an anti-digoxigenin antibody conjugated to horseradish peroxidase and thereafter incubation with HPPA or TMB, and thereby detecting anti-drug antibody in the serum or plasma sample (correlating the formed complex to the amount of the ADA in the sample).

29. The immunoassay according to any one of claims 1 to 28, wherein the sample is from a patient in need of a treatment with the drug to which the drug had been administered prior to obtaining the sample.

30. The immunoassay according to any one of claims 1 to 29, wherein the complexes are non-covalent complexes.

31. The immunoassay according to any one of claims 1 to 30, wherein the immunoassay comprises the following steps:
a) immobilizing the capture antibody on a solid surface, and optionally washing the surface after the immobilization step to remove unbound and non-specifically bound capture antibody,
b) incubating the immobilized capture antibody of step a) with a serum or plasma containing sample, which optionally has been diluted to have a concentration of the anti-drug antibody within the detection range of the immunoassay, to form a capture antibody-anti-drug antibody-complex, and optionally washing the surface after the incubation step to remove unbound and non-specifically bound sample,
c) incubating the capture antibody-anti-drug antibody-complex of step b) with a labelled tracer antibody to form a capture antibody-anti-drug antibody-tracer antibody complex, and optionally washing the surface after the incubation step to remove unbound and non-specifically bound tracer antibody,
d) incubating the capture antibody-anti-drug antibody-tracer antibody complex of step c) with an antibody specifically binding to the label of the tracer antibody conjugated to an enzyme to form a capture antibody-anti-drug antibody-tracer antibody-detection antibody complex, and optionally washing the surface after the incubation step to remove unbound and non-specifically bound detection antibody,
e) incubating the capture antibody-anti-drug antibody-tracer antibody-detection antibody complex of step d) with a colorless substrate of the enzyme that upon action of the enzyme on the substrate is converted to a colored reaction product and determining the optical density after a predefined period of time,
f) correlating the optical density determined in step e) with a calibration curve and thereby determining the amount of anti-drug antibody in the sample.


SEQUENCE LISTING (116 sequences)

SEQ ID NO: 1 - protein, 121 aa, synthetic construct
  REGION 1..121: note = CFA0305 VH
QSLEESGGDLVKPGASLAVTCTASGFSSSSSYYMCWVRQAPGKGLEWIGCIYTGSGATYYASWAKGRFTISKTSSTTVTLQMTSLTVADTATYFCASDGGYVTPTHAMYLWGPGTLVTVSS

SEQ ID NO: 2 - protein, 117 aa, synthetic construct
  REGION 1..117: note = CFA0307 VH
QSLEESGGGLVKPGASLTLTCKASGIDFSNFYYICWVRQAPGKGLDLIACIYTVSGYTYYASWAKGRLTISKTSSTTVTLQMTSLTAADTATYFCARDLHAGITNLWGPGTLVTVSS

SEQ ID NO: 3 - protein, 122 aa, synthetic construct
  REGION 1..122: note = CFA0366 VH
QSLEESGGDLVKPGASLSLTCTAAGLDFSSSYYMCWVRQAPGKGLEWIACIYAGSSGIIYYANWAKGRFTISRPSSTTVTLQMTSLTAADTATYFCATYPTYGDGGHAFNLWGPGTLVTVSS

SEQ ID NO: 4 - protein, 117 aa, synthetic construct
  REGION 1..117: note = CFA0501 VH
QSVEESGGRLVTPGTPLTLTCTASGFSLSSYYMNWVRQAPGKGLEWIGMIYGSGATYYASWAKGRFTISKTSSTTVDLKMTSLTTEDTATYFCARQIYSGDNNDNFWGPGTLVTVSS

SEQ ID NO: 5 - protein, 117 aa, synthetic construct; identical to SEQ ID NO: 4
  REGION 1..117: note = CFA0538 VH

SEQ ID NO: 6 - protein, 117 aa, synthetic construct
  REGION 1..117: note = CFA0599 VH
QSVEESGGRLVTPGTPLTLTCTVSGLSLSDNTMTWVRQAPGKGLEWIGIISFGGDAYYASWAKGRFPISKTSTTVDLRITSPTTEDTATYFCARVGAGNIFWYFDLWGPGTLVTVSS

SEQ ID NO: 7 - protein, 116 aa, synthetic construct
  REGION 1..116: note = CFA0666 VH
QSVEESGGRLVTPGTPLTLTCTVSGFSLSTYAMGWVRQAPGKGLEWIGTIDTGDNSFYANWAKGRFTISKTSTTVDLKITSPTAEDTATYFCARNDGSVYNLFNLWGPGTLVTVSS

SEQ ID NO: 8 - protein, 110 aa, synthetic construct
  REGION 1..110: note = CFA0672 VH
QSVEESGGRLVTPGTPLTLTCTASGFSLSGNAINWVRQAPGKGLEWIGCIYTGSDTTYYATWAKGRFTISKTSTTVDLKIASPTTEDTATYFCARGSGLWGPGTLVTVSS

SEQ ID NO: 9 - protein, 116 aa, synthetic construct
  REGION 1..116: note = CFA0675 VH
QSVEESGGRLVTPGTPLTLTCTVSGFSLSTYAMGWVRQAPGKGLEWIGTIDTGDNSFYANWAKGRFTISRTSTTVDLKITSPTAEDTATYFCARNDGSVYNLFNLWGPGTLVTVSS

SEQ ID NO: 10 - protein, 123 aa, synthetic construct
  REGION 1..123: note = 305LO5 VH
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 11 - protein, 110 aa, synthetic construct
  REGION 1..110: note = CFA0305 VL
DVVMTQTPASVSAAVGGTVTIKCQASQNIGSDLAWYQQKPGQPPRLLIYGASKLASGVSSRFSGSGSGKEFTLTISDLECADAVTYYCQCTFVGSSYGNAFGGGTEVVVK

SEQ ID NO: 12 - protein, 110 aa, synthetic construct
  REGION 1..110: note = CFA0307 VL
AIEMTQTPFSVSAAVGGTVTIKCQASQNIYSNLAWYQQKPGQRPKLLIYGASNLESGVPSRFKGSGSGTEYTLTISDLECADAATYYCLQGYSYSNVDDAFGGGTEVVVK

SEQ ID NO: 13 - protein, 112 aa, synthetic construct
  REGION 1..112: note = CFA0366 VL
AQVLTQTPSSVSAAVGGTVTINCQSSQSVYSSDYLSWYQQKPGQPPKLLIYEASKLASGVPPRFSGSGSGTQFTLTISGVQCDDAATYYCQGTYYSSGWYFAFGGGTEVVVK

SEQ ID NO: 14 - protein, 110 aa, synthetic construct
  REGION 1..110: note = CFA0501 VL
AYDMTQTPASVEVAVGGTVTINCQASESISNWLAWYQQKPGQRPKLLIYRASTLASGVSSRFKGSGSGTQFTLTISGVECDDAATYYCQQDYSSSNVDNTFGGGTKVVVE

SEQ ID NO: 15 - protein, 110 aa, synthetic construct
  REGION 1..110: note = CFA0538 VL
AYDMTQTPASVEVAVGGTVTINCQASESISNWLAWYQQKPGQRPKLLIYRASTLASGVSSRFKGSGSGTQFTLTISGVECDDAATYYCQQDYSSSNVDNTFGGGTTVVVE

SEQ ID NO: 16 - protein, 110 aa, synthetic construct
  REGION 1..110: note = CFA0599 VL
AYDMTQTPASVEVSVGGTVTIKCQASESIYSALAWYQQKPGQPPKLLIYSASTLASGVPSRFKGSGSGTEYTLTISDLECADAATYYCQQYYSSTNVHNSFGGGTTVVVE

SEQ ID NO: 17 - protein, 110 aa, synthetic construct
  REGION 1..110: note = CFA0666 VL
DVVMTQTPASVSAAVGGTVTIKCQASENIYSALSWYQQKPGLRPRLLIYYASTLASGVPSRFKGSRSGTEFTLTISDLECADAATYYCQQYYDINSVDNTFGGGTKVVVE

SEQ ID NO: 18 - protein, 111 aa, synthetic construct
  REGION 1..111: note = CFA0672 VL
DVVMTQTPASVSAPVGGTVTIKCQASQNIYSLLAWYQQKPGQPPKLLIYHTSDLASGVPSRFRGSGSGTEFTLTISDLECADAATYYCQCTAYGSSDVGGTFGGGTTVVVE

SEQ ID NO: 19 - protein, 110 aa, synthetic construct
  REGION 1..110: note = CFA0675 VL
DVVMTQTPASVSAAVGGTVTIKCQASENIYSALSWYQQKPGLRPRLLIYYASTLASGVPSRFKGSRSGTEFTLTISDLECADAASYYCQQYYDINSVDNTFGGGTKVVVE

SEQ ID NO: 20 - protein, 110 aa, synthetic construct
  REGION 1..110: note = 305LO5 VL
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 21 - protein, 117 aa, synthetic construct
  REGION 1..117: note = CFA0330 VH
QSLEESGGDLVEPGASLTLTCTASGFSWNSGYDMCWVRQAPGKGLEWIACIYTGSSANTAYANWAKGRFTISKTSSTTVTLQMTGLTAADTATYFCARHDDYFFDLWGPGTLVTVSS

SEQ ID NO: 22 - protein, 115 aa, synthetic construct
  REGION 1..115: note = CFA0341 VH
QSVEESGGRLVTPGTPLTLTCTVSGIDLSSYTMGWVRQAPGKGLECIGYIHSFGSTYYASWAKGRFTISKTSTTVDLEITSPTTEDTATYFCARDVGGSSGWDLWGQGTLVTVSS

SEQ ID NO: 23 - protein, 116 aa, synthetic construct
  REGION 1..116: note = CFA0329 VH
QSLEESGGGLVKPGASLTLTCTASGFSFSSGYYMCWVRQAPGKGLEWIGCIGTISDSTYYASWAKGRFPISKASSTTVTLQMTSLTAADTATYFCARDPYSYGDLWGPGTLVTVSS

SEQ ID NO: 24 - protein, 114 aa, synthetic construct
  REGION 1..114: note = CFA0330 VL
DVVMTQTPASVSESVGGTVTINCQASQSIGNNLAWYQQKPGQPPKLLIYDASNLASGVPSRFKGSRSGTEPTLTNTLTISGVQCADVATYYCQSGWYGNSYVAAFGGGTEVVVK

SEQ ID NO: 25 - protein, 112 aa, synthetic construct
  REGION 1..112: note = CFA0341 VL
DVVMTQTPSSVSAAVGGTVTIKCQASENIYSNLAWYQQKPGQPPKRLIYDASDLASGVPSRFSGSGYGTQFTLTISGVQCEDAATYYCQANYDSTSSSFGNVFGGGTEVVVK

SEQ ID NO: 26 - protein, 110 aa, synthetic construct
  REGION 1..110: note = CFA0329 VL
DLVMTQTPASVSEPVGGTVTIKCQASQSIGTYLAWYQQKPGQPPKLLIYRASTLASGVPSRFKGSGSGTQFTLTISDLECADAATYYCLGGYYGFSYGNAFGGGTEVVVK

SEQ ID NO: 27 - protein, 325 aa, synthetic construct
  REGION 1..325: note = F760G4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRGGPKVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 28 - protein, 325 aa, synthetic construct
  REGION 1..325: note = F939G4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRGGPKVFLFPPKPKDTLYISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHYHVTQKSLSLSL

SEQ ID NO: 29 - protein, 325 aa, synthetic construct
  REGION 1..325: note = SG402
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRGGPKVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 30 - protein, 1676 aa, synthetic construct
  REGION 1..1676: note = human C5
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 31 - protein, 673 aa, synthetic construct
  REGION 1..673: note = human C5, beta chain
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEIL

SEQ ID NO: 32 - protein, 105 aa, synthetic construct
  REGION 1..105: note = human C5, MG1
EQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNG

SEQ ID NO: 33 - protein, 101 aa, synthetic construct
  REGION 1..101: note = human C5, MG2
FLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLP

SEQ ID NO: 34 - protein, 225 aa, synthetic construct
  REGION 1..225: note = human C5, MG1-MG2
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLP

SEQ ID NO: 35 - protein, 1676 aa, synthetic construct
  REGION 1..1676: note = cynomolgus C5
MGLLGILCFLIFLGKTWGQEQTYVISAPKIFRVGASENIVIQVYGYTEAFDATISIKSYPDKKFSYSSGHVHLSSENKFQNSAVLTIQPKQLPGGQNQVSYVYLEVVSKHFSKSKKIPITYDNGFLFIHTDKPVYTPDQSVKVRVYSLNDDLKPAKRETVLTFIDPEGSEIDMVEEIDHIGIISFPDFKIPSNPRYGMWTIQAKYKEDFSTTGTAFFEVKEYVLPHFSVSVEPESNFIGYKNFKNFEITIKARYFYNKVVTEADVYITFGIREDLKDDQKEMMQTAMQNTMLINGIAEVTFDSETAVKELSYYSLEDLNNKYLYIAVTVIESTGGFSEEAEIPGIKYVLSPYKLNLVATPLFLKPGIPYSIKVQVKDALDQLVGGVPVTLNAQTIDVNQETSDLEPRKSVTRVDDGVASFVVNLPSGVTVLEFNVKTDAPDLPDENQAREGYRAIAYSSLSQSYLYIDWTDNHKALLVGEYLNIIVTPKSPYIDKITHYNYLILSKGKIIHFGTREKLSDASYQSINIPVTQNMVPSSRLLVYYIVTGEQTAELVSDSVWLNIEEKCGNQLQVHLSPDADTYSPGQTVSLNMVTGMDSWVALTAVDSAVYGVQRRAKKPLERVFQFLEKSDLGCGAGGGLNNANVFHLAGLTFLTNANADDSQENDEPCKEIIRPRRMLQEKIEEIAAKYKHLVVKKCCYDGVRINHDETCEQRAARISVGPRCVKAFTECCVVASQLRANNSHKDLQLGRLHMKTLLPVSKPEIRSYFPESWLWEVHLVPRRKQLQFALPDSVTTWEIQGVGISNSGICVADTIKAKVFKDVFLEMNIPYSVVRGEQVQLKGTVYNYRTSGMQFCVKMSAVEGICTSESPVIDHQGTKSSKCVRQKVEGSSNHLVTFTVLPLEIGLQNINFSLETSFGKEILVKSLRVVPEGVKRESYSGITLDPRGIYGTISRRKEFPYRIPLDLVPKTEIKRILSVKGLLVGEILSAVLSREGINILTHLPKGSAEAELMSVVPVFYVFHYLETGNHWNIFHSDPLIEKRNLEKKLKEGMVSIMSYRNADYSYSVWKGGSASTWLTAFALRVLGQVHKYVEQNQNSICNSLLWLVENYQLDNGSFKENSQYQPIKLQGTLPVEARENSLYLTAFTVIGIRKAFDICPLVKINTALIKADTFLLENTLPAQSTFTLAISAYALSLGDKTHPQFRSIVSALKREALVKGNPPIYRFWKDSLQHKDSSVPNTGTARMVETTAYALLTSLNLKDINYVNPIIKWLSEEQRYGGGFYSTQDTINAIEGLTEYSLLVKQLRLNMDIDVAYKHKGPLHNYKMTDKNFLGRPVEVLLNDDLVVSTGFGSGLATVHVTTVVHKTSTSEEVCSFYLKIDTQDIEASHYRGYGNSDYKRIVACASYKPSKEESSSGSSHAVMDISLPTGINANEEDLKALVEGVDQLFTDYQIKDGHVILQLNSIPSSDFLCVRFRIFELFEVGFLSPATFTVYEYHRPDKQCTMFYSTSNIKIQKVCEGATCKCIEADCGQMQKELDLTISAETRKQTACNPEIAYAYKVIITSITTENVFVKYKATLLDIYKTGEAVAEKDSEITFIKKVTCTNAELVKGRQYLIMGKEALQIKYNFTFRYIYPLDSLTWIEYWPRDTTCSSCQAFLANLDEFAEDIFLNGC

SEQ ID NO: 36 - protein, 6 aa, synthetic construct
  REGION 1..6: note = CFA0305 HVR-H1
SSYYMC

SEQ ID NO: 37 - protein, 6 aa, synthetic construct
  REGION 1..6: note = CFA0307 HVR-H1
NFYYIC

SEQ ID NO: 38 - protein, 6 aa, synthetic construct
  REGION 1..6: note = CFA0366 HVR-H1
SSYYMC

SEQ ID NO: 39 - protein, 6 aa, synthetic construct
  REGION 1..6: note = CFA0501 HVR-H1
SSYYMN

SEQ ID NO: 40 - protein, 6 aa, synthetic construct
  REGION 1..6: note = CFA0538 HVR-H1
SSYYMN

SEQ ID NO: 41 - protein, 5 aa, synthetic construct
  REGION 1..5: note = CFA0599 HVR-H1
DNTMT

SEQ ID NO: 42 - protein, 5 aa, synthetic construct
  REGION 1..5: note = CFA0666 HVR-H1
TYAMG

SEQ ID NO: 43 - protein, 5 aa, synthetic construct
  REGION 1..5: note = CFA0672 HVR-H1
GNAIN

SEQ ID NO: 44 - protein, 5 aa, synthetic construct
  REGION 1..5: note = CFA0675 HVR-H1
TYAMG

SEQ ID NO: 45 - protein, 6 aa, synthetic construct
  REGION 1..6: note = 305LO5 HVR-H1
SSYYVA

SEQ ID NO: 46 - protein, 17 aa, synthetic construct
  REGION 1..17: note = CFA0305 HVR-H2
CIYTGSGATYYASWAKG

SEQ ID NO: 47 - protein, 17 aa, synthetic construct
  REGION 1..17: note = CFA0307 HVR-H2
CIYTVSGYTYYASWAKG

SEQ ID NO: 48 - protein, 18 aa, synthetic construct
  REGION 1..18: note = CFA0366 HVR-H2
CIYAGSSGIIYYANWAKG

SEQ ID NO: 49 - protein, 16 aa, synthetic construct
  REGION 1..16: note = CFA0501 HVR-H2
MIYGSGATYYASWAKG

SEQ ID NO: 50 - protein, 16 aa, synthetic construct
  REGION 1..16: note = CFA0538 HVR-H2
MIYGSGATYYASWAKG

SEQ ID NO: 51 - protein, 16 aa, synthetic construct
  REGION 1..16: note = CFA0599 HVR-H2
IISFGGDAYYASWAKG

SEQ ID NO: 52 - protein, 16 aa, synthetic construct
  REGION 1..16: note = CFA0666 HVR-H2
TIDTGDNSFYANWAKG

SEQ ID NO: 53 - protein, 17 aa, synthetic construct
  REGION 1..17: note = CFA0672 HVR-H2
CIYTGSDTTYYATWAKG

SEQ ID NO: 54 - protein, 16 aa, synthetic construct
  REGION 1..16: note = CFA0675 HVR-H2
TIDTGDNSFYANWAKG

SEQ ID NO: 55 - protein, 17 aa, synthetic construct
  REGION 1..17: note = 305LO5 HVR-H2
AIYTGSGATYKASWAKG

SEQ ID NO: 56 - protein, 13 aa, synthetic construct
  REGION 1..13: note = CFA0305 HVR-H3
DGGYVTPTHAMYL

SEQ ID NO: 57 - protein, 9 aa, synthetic construct
  REGION 1..9: note = CFA0307 HVR-H3
DLHAGITNL

SEQ ID NO: 58 - protein, 13 aa, synthetic construct
  REGION 1..13: note = CFA0366 HVR-H3
YPTYGDGGHAFNL

SEQ ID NO: 59 - protein, 11 aa, synthetic construct
  REGION 1..11: note = CFA0501 HVR-H3
QIYSGDNNDNF

SEQ ID NO: 60 - protein, 11 aa, synthetic construct
  REGION 1..11: note = CFA0538 HVR-H3
QIYSGDNNDNF

SEQ ID NO: 61 - protein, 12 aa, synthetic construct
  REGION 1..12: note = CFA0599 HVR-H3
VGAGNIFWYFDL

SEQ ID NO: 62 - protein, 11 aa, synthetic construct
  REGION 1..11: note = CFA0666 HVR-H3
NDGSVYNLFNL

SEQ ID NO: 63 - protein, 4 aa, synthetic construct
  REGION 1..4: note = CFA0672 HVR-H3
GSGL

SEQ ID NO: 64 - protein, 11 aa, synthetic construct
  REGION 1..11: note = CFA0675 HVR-H3
NDGSVYNLFNL

SEQ ID NO: 65 - protein, 13 aa, synthetic construct
  REGION 1..13: note = 305LO5 HVR-H3
DGGYDYPTHAMHY

SEQ ID NO: 66 - protein, 11 aa, synthetic construct
  REGION 1..11: note = CFA0305 HVR-L1
QASQNIGSDLA

SEQ ID NO: 67 - protein, 11 aa, synthetic construct
  REGION 1..11: note = CFA0307 HVR-L1
QASQNIYSNLA

SEQ ID NO: 68 - protein, 13 aa, synthetic construct
  REGION 1..13: note = CFA0366 HVR-L1
QSSQSVYSSDYLS

SEQ ID NO: 69 - protein, 11 aa, synthetic construct
  REGION 1..11: note = CFA0501 HVR-L1
QASESISNWLA

SEQ ID NO: 70 - protein, 11 aa, synthetic construct
  REGION 1..11: note = CFA0538 HVR-L1
QASESISNWLA

SEQ ID NO: 71 - protein, 11 aa, synthetic construct
  REGION 1..11: note = CFA0599 HVR-L1
QASESIYSALA

SEQ ID NO: 72 - protein, 11 aa, synthetic construct
  REGION 1..11: note = CFA0666 HVR-L1
QASENIYSALS

SEQ ID NO: 73 - protein, 11 aa, synthetic construct
  REGION 1..11: note = CFA0672 HVR-L1
QASQNIYSLLA

SEQ ID NO: 74 - protein, 11 aa, synthetic construct
  REGION 1..11: note = CFA0675 HVR-L1
QASENIYSALS

SEQ ID NO: 75 - protein, 11 aa, synthetic construct
  REGION 1..11: note = 305LO5 HVR-L1
QASQNIGSSLA

SEQ ID NO: 76 - protein, 7 aa, synthetic construct
  REGION 1..7: note = CFA0305 HVR-L2
GASKLAS

SEQ ID NO: 77 - protein, 7 aa, synthetic construct
  REGION 1..7: note = CFA0307 HVR-L2
GASNLES

SEQ ID NO: 78 - protein, 7 aa, synthetic construct
  REGION 1..7: note = CFA0366 HVR-L2
EASKLAS

SEQ ID NO: 79 - protein, 7 aa, synthetic construct
  REGION 1..7: note = CFA0501 HVR-L2
RASTLAS

SEQ ID NO: 80 - protein, 7 aa, synthetic construct
  REGION 1..7: note = CFA0538 HVR-L2
RASTLAS

SEQ ID NO: 81 - protein, 7 aa, synthetic construct
  REGION 1..7: note = CFA0599 HVR-L2
SASTLAS

SEQ ID NO: 82 - protein, 7 aa, synthetic construct
  REGION 1..7: note = CFA0666 HVR-L2
YASTLAS

SEQ ID NO: 83 - protein, 7 aa, synthetic construct
  REGION 1..7: note = CFA0672 HVR-L2
HTSDLAS

SEQ ID NO: 84 - protein, 7 aa, synthetic construct
  REGION 1..7: note = CFA0675 HVR-L2
YASTLAS

SEQ ID NO: 85 - protein, 7 aa, synthetic construct
  REGION 1..7: note = 305LO5 HVR-L2
GASKTHS

SEQ ID NO: 86 - protein, 12 aa, synthetic construct
  REGION 1..12: note = CFA0305 HVR-L3
QCTFVGSSYGNA

SEQ ID NO: 87 - protein, 12 aa, synthetic construct
  REGION 1..12: note = CFA0307 HVR-L3
LQGYSYSNVDDA

SEQ ID NO: 88 - protein, 12 aa, synthetic construct
  REGION 1..12: note = CFA0366 HVR-L3
QGTYYSSGWYFA

SEQ ID NO: 89 - protein, 12 aa, synthetic construct
  REGION 1..12: note = CFA0501 HVR-L3
QQDYSSSNVDNT

SEQ ID NO: 90 - protein, 12 aa, synthetic construct
  REGION 1..12: note = CFA0538 HVR-L3
QQDYSSSNVDNT

SEQ ID NO: 91 - protein, 12 aa, synthetic construct
  REGION 1..12: note = CFA0599 HVR-L3
QQYYSSTNVHNS

SEQ ID NO: 92 - protein, 12 aa, synthetic construct
  REGION 1..12: note = CFA0666 HVR-L3
QQYYDINSVDNT

SEQ ID NO: 93 - protein, 13 aa, synthetic construct
  REGION 1..13: note = CFA0672 HVR-L3
QCTAYGSSDVGGT

SEQ ID NO: 94 - protein, 12 aa, synthetic construct
  REGION 1..12: note = CFA0675 HVR-L3
QQYYDINSVDNT

SEQ ID NO: 95 - protein, 12 aa, synthetic construct
  REGION 1..12: note = 305LO5 HVR-L3
QSTKVGSSYGNH

SEQ ID NO: 96 - protein, 1680 aa, synthetic construct
  REGION 1..1680: note = mouse C5
MGLWGILCLLIFLDKTWGQEQTYVISAPKILRVGSSENVVIQVHGYTEAFDATLSLKSYPDKKVTFSSGYVNLSPENKFQNAALLTLQPNQVPREESPVSHVYLEVVSKHFSKSKKIPITYNNGILFIHTDKPVYTPDQSVKIRVYSLGDDLKPAKRETVLTFIDPEGSEVDIVEENDYTGIISFPDFKIPSNPKYGVWTIKANYKKDFTTTGTAYFEIKEYVLPRFSVSIELERTFIGYKNFKNFEITVKARYFYNKVVPDAEVYAFFGLREDIKDEEKQMMHKATQAAKLVDGVAQISFDSETAVKELSYNSLEDLNNKYLYIAVTVTESSGGFSEEAEIPGVKYVLSPYTLNLVATPLFVKPGIPFSIKAQVKDSLEQAVGGVPVTLMAQTVDVNQETSDLETKRSITHDTDGVAVFVLNLPSNVTVLKFEIRTDDPELPEENQASKEYEAVAYSSLSQSYIYIAWTENYKPMLVGEYLNIMVTPKSPYIDKITHYNYLILSKGKIVQYGTREKLFSSTYQNINIPVTQNMVPSARLLVYYIVTGEQTAELVADAVWINIEEKCGNQLQVHLSPDEYVYSPGQTVSLDMVTEADSWVALSAVDRAVYKVQGNAKRAMQRVFQALDEKSDLGCGAGGGHDNADVFHLAGLTFLTNANADDSHYRDDSCKEILRSKRNLHLLRQKIEEQAAKYKHSVPKKCCYDGARVNFYETCEERVARVTIGPLCIRAFNECCTIANKIRKESPHKPVQLGRIHIKTLLPVMKADIRSYFPESWLWEIHRVPKRKQLQVTLPDSLTTWEIQGIGISDNGICVADTLKAKVFKEVFLEMNIPYSVVRGEQIQLKGTVYNYMTSGTKFCVKMSAVEGICTSGSSAASLHTSRPSRCVFQRIEGSSSHLVTFTLLPLEIGLHSINFSLETSFGKDILVKTLRVVPEGVKRESYAGVILDPKGIRGIVNRRKEFPYRIPLDLVPKTKVERILSVKGLLVGEFLSTVLSKEGINILTHLPKGSAEAELMSIAPVFYVFHYLEAGNHWNIFYPDTLSKRQSLEKKIKQGVVSVMSYRNADYSYSMWKGASASTWLTAFALRVLGQVAKYVKQDENSICNSLLWLVEKCQLENGSFKENSQYLPIKLQGTLPAEAQEKTLYLTAFSVIGIRKAVDICPTMKIHTALDKADSFLLENTLPSKSTFTLAIVAYALSLGDRTHPRFRLIVSALRKEAFVKGDPPIYRYWRDTLKRPDSSVPSSGTAGMVETTAYALLASLKLKDMNYANPIIKWLSEEQRYGGGFYSTQDTINAIEGLTEYSLLLKQIHLDMDINVAYKHEGDFHKYKVTEKHFLGRPVEVSLNDDLVVSTGYSSGLATVYVKTVVHKISVSEEFCSFYLKIDTQDIEASSHFRLSDSGFKRIIACASYKPSKEESTSGSSHAVMDISLPTGIGANEEDLRALVEGVDQLLTDYQIKDGHVILQLNSIPSRDFLCVRFRIFELFQVGFLNPATFTVYEYHRPDKQCTMIYSISDTRLQKVCEGAACTCVEADCAQLQAEVDLAISADSRKEKACKPETAYAYKVRITSATEENVFVKYTATLLVTYKTGEAADENSEVTFIKKMSCTNANLVKGKQYLIMGKEVLQIKHNFSFKYIYPLDSSTWIEYWPTDTTCPSCQAFVENLNNFAEDLFLNSCE

SEQ ID NO: 97 - protein, 123 aa, synthetic construct [sequence omitted]

SEQ ID NO: 98 - protein, 123 aa, synthetic construct
  REGION 1..123: note = 305LO16 VH
QVQLVESGGGLVQPGRSLRLSCAASGFTVHSSYYMAWVRQAPGKGLEWVGAIFTGSGAEYKAEWVKGRFTISRDNSKNTLYLQMNSLRAEDTAVYYCASDAGYDYPTHAMHYWGRGTLVTVSS

SEQ ID NO: 99 - protein, 123 aa, synthetic construct
  REGION 1..123: note = 305LO18 VH
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGRGTLVTVSS

SEQ ID NO: 100 - protein, 123 aa, synthetic construct
  REGION 1..123: note = 305LO19, 20, 22 VH
QVQLVESGGGLVQPGGSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSS

SEQ ID NO: 101 - protein, 123 aa, synthetic construct
  REGION 1..123: note = 305LO23 VH
QVQLVESGGGLVQPGGSLRLSCAASGFTVHSSYYMAWVRQAPGKGLEWVSGIFTGSGATYKAEWAKGRVTISKDTSKNQVVLTMTNMDPVDTATYYCASDAGYDYPTHAMHYWGQGTLVTVSS

SEQ ID NO: 102 - protein, 110 aa, synthetic construct [sequence omitted]

SEQ ID NO: 103 - protein, 110 aa, synthetic construct
  REGION 1..110: note = 305LO20 VL
DIQMTQSPSSLSASVGDRVTITCRASQGISSSLAWYQQKPGQAPRLLIYGASETESGVPSRFSGSGSGTDFTLTISSLQPEDFATYYCQNTKVGSSYGNTFGGGTKVEIK

SEQ ID NO: 104 - protein, 110 aa, synthetic construct
  REGION 1..110: note = 305LO22, 23 VL
DIQMTQSPSSLSASVGDRVTITCRASQGISSSLAWYQQKPGQAPRLLIYGASTTQSGVPSRFSGSGSGTDFTLTISSLQPEDFATYYCQNTKVGSSYGNTFGGGTKVEIK

SEQ ID NO: 105 - protein, 328 aa, synthetic construct
  REGION 1..328: note = SG115
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 106 - protein, 325 aa, synthetic construct
  REGION 1..325: note = SG422
ASTKGPSVFPLAPCSRSTSGGTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 107 - protein, 325 aa, synthetic construct
  REGION 1..325: note = SG429
ASTKGPSVFPLAPCSRSTSGGTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHAHYTRKELSLSP

SEQ ID NO: 108 - protein, 6 aa, synthetic construct
  REGION 1..6: note = 305LO15, 16, 18, 19, 20, 22, 23 HVR-H1
SSYYMA

SEQ ID NO: 109 - protein, 17 aa, synthetic construct
  REGION 1..17: note = 305LO15, 18, 19, 20, 22 HVR-H2
AIFTGSGAEYKAEWAKG

SEQ ID NO: 110 - protein, 17 aa, synthetic construct
  REGION 1..17: note = 305LO16 HVR-H2
AIFTGSGAEYKAEWVKG

SEQ ID NO: 111 - protein, 17 aa, synthetic construct
  REGION 1..17: note = 305LO23 HVR-H2
GIFTGSGATYKAEWAKG

SEQ ID NO: 112 - protein, 13 aa, synthetic construct
  REGION 1..13: note = 305LO15, 16, 18, 19, 20, 22, 23 HVR-H3
DAGYDYPTHAMHY

SEQ ID NO: 113 - protein, 11 aa, synthetic construct
  REGION 1..11: note = 305LO15, 16, 18, 19, 20, 22, 23 HVR-L1
RASQGISSSLA

SEQ ID NO: 114 - protein, 7 aa, synthetic construct
  REGION 1..7: note = 305LO15, 16, 18, 19, 20 HVR-L2
GASETES

SEQ ID NO: 115 - protein, 7 aa, synthetic construct
  REGION 1..7: note = 305LO22, 23 HVR-L2
GASTTQS

SEQ ID NO: 116 - protein, 12 aa, synthetic construct
  REGION 1..12: note = 305LO15, 16, 18, 19, 20, 22, 23 HVR-L3
QNTKVGSSYGNT